(19) United States
(12) Patent Application Publication
(10) Pub. No.: US 2024/0342427 A1
(43) Pub. Date: Oct. 17, 2024

(54) VENTILATION ARRANGEMENT AND VENTILATION PROCESS WITH A COMPENSATION OF VIBRATIONS IN A VALVE

(71) Applicant: Drägerwerk AG & Co. KGaA, Lübeck (DE)

(72) Inventors: Hans-Ullrich HANSMANN, Lübeck (DE); Sebastian SCHRÖTER, Lübeck (DE); Jöran BLENDERMANN, Lübeck (DE)

(21) Appl. No.: 18/632,568

(22) Filed: Apr. 11, 2024

(30) Foreign Application Priority Data
Apr. 13, 2023 (DE) 10 2023 109 254.5
Apr. 13, 2023 (DE) 10 2023 109 255.3

(51) Int. Cl. A61M 16/20; A61M 16/00

(52) U.S. Cl. CPC A61M 16/202; A61M 16/0003; A61M 2205/3327; A61M 2205/42

(57) ABSTRACT

A ventilation arrangement and a ventilation process provide ventilation for a patient. A first segment of a fluid guiding unit connects a fluid delivery unit to a valve (10). A second segment connects the valve (10) to a coupling unit on the patient side. A position of a valve body (19) of the valve (10) relative to a valve body seat (18) depends on an inlet pressure (P2) and on a control pressure (P1) and influences the volume flow (Vol′) through the second segment. An actuator (15) changes the control pressure (P1). A control signal (Sig_{con}) for the actuator (15) is generated with the objective of ensuring that the pressure or volume flow (Vol′) in the second segment assumes a predetermined value. A compensation signal (Sig_{comp}) for the actuator (15) is generated with the objective of preventing the valve body (19) from vibrating relative to the valve body seat (18).

### CROSS REFERENCE TO RELATED APPLICATIONS

This application claims the benefit of priority under 35 U.S.C. § 119 of German Applications 10 2023 109 254.5, filed Apr. 13, 2023, and 10 2023 109 255.3, filed Apr. 13, 2023, the entire contents of which are incorporated herein by reference.

### TECHNICAL FIELD

The invention relates to a ventilation arrangement and a ventilation process by means of which a patient can be artificially ventilated (ventilated).

### BACKGROUND

Just like many ventilation arrangements known from the prior art, the ventilation arrangement according to the invention comprises a ventilator (respirator), an inspiratory fluid guide unit and a patient-side coupling unit. The patient-side coupling unit is at least temporarily arranged on and/or in and/or at the body of a patient who is being artificially ventilated. The inspiratory fluid guide unit connects the ventilator to the patient-side coupling unit. The ventilator ejects a gas mixture, the gas mixture comprising oxygen and, in one embodiment, at least one anesthetic. Preferably, the ventilator performs a sequence of ventilation strokes and ejects in each ventilation stroke a quantity of the gas mixture. The expelled gas mixture is passed (flows) through the inspiratory fluid guide unit to the patient-side coupling unit, and the patient can inhale the gas mixture.

The volume flow of the gas mixture to the patient-side coupling unit and/or the pressure in the inspiratory fluid guide unit should often follow a predefined time course (time curve, chronological sequence). To achieve this goal, a valve is arranged in the inspiratory fluid guide unit. An input of the valve is connected to the ventilator via a segment of the inspiratory fluid guide unit, and an output is connected to the patient-side coupling unit via another segment. The valve comprises a valve body seat and a valve body that can be moved relative to the valve body seat. By moving the valve body relative to the valve body seat, the volume flow and/or the pressure downstream of the valve are influenced and thereby controlled.

### SUMMARY

It is an object of the invention to provide a ventilation arrangement comprising a ventilator, an inspiratory fluid guide unit and a patient-side coupling unit, wherein a setting parameter of the inspiratory fluid guide unit, in particular the pressure and/or the volume flow, can be changed relatively quickly with the aid of the valve and wherein the risk of an undesirable side effect occurring is to be reduced. Furthermore, the invention is based on the task of providing a corresponding ventilation process.

The task is solved by a ventilation arrangement with features according to the invention and by a ventilation process with features according to the invention. Advantageous embodiments are disclosed. Advantageous embodiments of the ventilation arrangement according to the invention are, where appropriate, also advantageous embodiments of the ventilation process according to the invention and vice versa.

The ventilation arrangement according to the invention and the ventilation process according to the invention are configured to be used for artificial (mechanical) ventilation of a patient. During artificial ventilation, a patient-side coupling unit is arranged on and/or in and/or at the patient's body. A breathing mask and a tube are two examples of a patient-side coupling unit. The patient-side coupling unit can be a component of the ventilation arrangement.

The ventilation arrangement according to the invention comprises a fluid delivery unit. A fluid delivery unit is understood to be a device which is capable of delivering (conveying) a fluid. A blower, a pump, and a piston-cylinder unit are three examples of a fluid delivery unit.

An inspiratory fluid guide unit of the ventilation arrangement connects the fluid delivery unit to the patient-side coupling unit. A fluid guide unit is a component that is able to guide a fluid along a predetermined trajectory and ideally completely prevents part of the fluid from leaving this trajectory. A smooth hose, a corrugated hose, and a tube are three examples of a fluid guide unit.

Furthermore, the ventilation arrangement comprises a valve arrangement with at least one valve. A first segment of the inspiratory fluid guide unit connects the fluid delivery unit to the valve arrangement. A second segment of the inspiratory fluid guide unit connects the valve arrangement to the patient-side coupling unit. The two segments are connected in series. The fluid delivery unit is able to generate a flow of gas through the first segment. The gas flows from the fluid delivery unit through the first segment, through the valve arrangement and through the second segment to the patient-side coupling unit. The gas is a gas mixture which comprises oxygen and, in one embodiment, additionally at least one anesthetic agent and can be inhaled by a human being.

The or each valve of the valve arrangement is arranged between the first segment and the second segment. The or each valve comprises a respective valve body seat and a valve body. The valve body is movable relative to the valve body seat. The position of the valve body relative to the valve body seat depends on an inlet pressure (upstream pressure) and a control pressure. The inlet pressure is caused by the fluid delivery unit. The inlet pressure and the control pressure influence the relative position of the valve body, and the position of the valve body influences at least one setting parameter describing a pneumatic property of the second segment. Two examples of a setting parameter are a volume flow through the second segment and a pressure in the second segment. If the valve body is in circumferential contact with the valve body seat, the fluid connection through the valve is ideally completely prevented. The inlet pressure aims at moving the valve body away from the valve body seat. The control pressure aims at moving the valve body towards the valve body seat and press it against the valve body seat.

The ventilation arrangement comprises an actuator arrangement with at least one controllable actuator. A respective actuator of the actuator arrangement is assigned to the or each valve of the valve arrangement. The actuator assigned to a valve is able to contribute to the generation of the control pressure acting on this valve. In one embodiment, the control pressure of a valve is produced exclusively by the assigned actuator. It is also possible that the valve body is configured to be elastic and exerts a restoring spring force when deflected from a rest position, which spring force also contributes to the control pressure.

A signal-processing control unit of the ventilation arrangement is able to generate a control signal and a compensation signal and to control the or each actuator of the actuator arrangement. The control of the actuator arrangement by the control unit depends on the control signal, the compensation signal, or a superposition of these two signals. The controlled actuator arrangement contributes to the generation of the control pressure. It is possible that the actuator arrangement comprises several actuators and the control unit generates a respective control signal and a respective compensation signal for every actuator of the actuator arrangement.

The control unit is configured to generate the two signals with the following two control objectives (gains):
Due to the control with the activation signal, at least one setting parameter assumes a predefined value. The setting parameter describes a pneumatic property of the second segment. The pneumatic property is, in particular, a volume flow through the second segment or a pressure in the second segment. The predetermined value often results from a predetermined required time course of the setting parameter.
Due to the control with the compensation signal, vibration of the valve body relative to the valve body seat is completely or at least partially prevented.

The ventilation process according to the invention is carried out using such a ventilation device and comprises the corresponding steps.

The volume flow (volume flow rate) through a fluid guide unit is understood to be the volume per time unit that flows through the fluid guide unit, the volume flow being measured in l/min, for example.

Thanks to the valve arrangement, in many cases it is possible to ensure that the setting parameter assumes a predetermined setpoint value and, in particular, follows a predetermined required time course. Thanks to the valve, it is not necessary to control the fluid delivery unit directly. It is often possible for the fluid delivery unit to generate a constant volume flow and/or a constant pressure over time, even if the setting parameter is to vary over time. In many cases, this makes it easier to mechanically and/or electrically and/or pneumatically construct the fluid delivery unit than if the output of the fluid delivery unit had to follow a predetermined time course.

With the valves available today, a rapid change in a setting parameter (often several 100 Hz) can be achieved. However, the inventors have internally observed the following event: The rapid change often causes the valve body to vibrate relative to the valve body seat (“it bounces”), especially if relatively small volume flows have to be changed quickly at a relatively low pressure and if the valve body is therefore relatively close to the valve body seat. These vibrations are often caused by a rapid exchange of energy between pneumatic kinetics (here: volume flow of the flowing gas) and potential (pressure of the gas). The vibrations can lead to disturbing noises (a “humming”).

The compensation signal can help to reduce these disruptive vibrations, ideally eliminating them completely.

Various configurations of how the compensation signal is generated are possible.

In one embodiment, the compensation signal oscillates with at least one frequency. The or each frequency at which the compensation signal oscillates lies within a predetermined frequency band and is preferably sufficiently broadband. The frequency is not necessarily adapted to a frequency at which the valve body vibrates. The control unit can change the frequency. This embodiment often requires a relatively low computing capacity and often no sensor for measuring the vibrations because it is not necessary to measure unwanted vibrations. In particular, if the frequency of the compensation signal is changed quickly, it is often possible to prevent a disturbing vibration from building up as a wave.

In another embodiment, the control unit automatically adjusts the compensation signal to an undesired oscillation. According to this embodiment, the ventilation arrangement comprises an oscillation system. Gas flows through this oscillation system. The oscillation system comprises the valve assembly and optionally all or at least part of the inspiratory fluid guide unit. At least one parameter of the gas flowing through the oscillation system oscillates. This parameter is denoted as oscillation parameter. The oscillation parameter is in particular the pressure or the volume flow. The oscillation parameter has an oscillation signal component with the following properties or can have such an oscillation signal component: The oscillation signal component causes the valve body to vibrate relative to the valve body seat, or can at least contribute to such oscillation. The oscillation signal component oscillates with an oscillation signal component frequency and an oscillation signal component amplitude.

The control unit generates the compensation signal in such a way that the compensation signal at least partially compensates for this oscillation signal component, ideally compensating it completely. The control unit generates the compensation signal with the following control objective (gain): The compensation signal has the oscillation signal component frequency and the oscillation signal component amplitude and is phase-shifted with the oscillation of the oscillation signal component. Ideally the phase shift effects that the compensation signal eliminates the oscillation signal component. This match can usually only be achieved approximately.

In many cases, this configuration suppresses the vibration better than other possible configurations for generating the compensation signal. This configuration makes it possible to adapt the compensation signal to different situations during artificial ventilation, in particular to changes in a desired volume flow and/or a desired pressure or also to a pneumatic or mechanical change in the inspiratory fluid guide unit or the valve arrangement.

Preferably, the ventilation arrangement comprises at least one parameter sensor. The or each parameter sensor is capable of measuring an indicator of a pneumatic parameter. In one embodiment, the measured parameter is a pneumatic parameter of the second segment, in particular the setting parameter. In another embodiment, the measured parameter is a pneumatic parameter of the first segment, in particular a volume flow through the first segment or a pressure in the first segment. For example, the parameter sensor measures an indicator of the volume flow through the second segment or an indicator of the pressure in the second segment. The parameter sensor generates and delivers a parameter signal that describes the time course of the measured pneumatic parameter. The control unit is able to generate the compensation signal using the parameter signal in such a way that the following objective is achieved: The compensation signal has the oscillation signal component frequency and the oscillation signal component amplitude and is phase-shifted with the oscillation of the oscillation signal component.

The control unit is able to automatically adapt the compensation signal to the determined signal component oscillation frequency, the determined signal component oscillation amplitude and optionally the determined phase position (phasing). This adaptation is carried out with the following objective: Controlling the actuator arrangement with the compensation signal causes the oscillation of the oscillation signal component to be at least partially compensated. This also dampens the vibration of the valve body, ideally preventing vibrations completely.

The implementation with the parameter sensor eliminates the need to directly measure a vibration of the valve body, in particular to make a measurement with a microphone. Such a direct measurement is often relatively prone to errors.

According to a preferred implementation of the embodiment just described, the control unit is able to determine the signal component oscillation frequency, the oscillation signal component amplitude and optionally the phase position of the oscillation of the oscillation signal component as follows: The control unit applies a signal analysis process to the setting parameter signal and determines the frequency, the amplitude, and the phase position of the oscillation-more precisely: the frequency, the amplitude, and the phase position of a signal component in the parameter signal which describes the oscillation.

A different implementation avoids the need to measure the phase position directly. According to the different implementation, on a trial base different values are used for the phase position of the interfering (vibration-causing) signal component. Each phase position leads to one amplitude of the signal component. The phase position that leads to the smallest amplitude is subsequently used. By this way a target function, in this case the amplitude, is minimized.

Another implementation avoids the need to carry out a signal analysis. According to these other implementation, the parameter signal is fed back. An oscillating signal component is determined in the parameter signal, preferably by suitable filtering of the setting parameter signal, for example by means of a high-pass filter, which filters out low-frequency signal components and non-oscillating signal components from the parameter signal. The result of the filtering provides—optionally after signal processing—the oscillation signal component. The control unit generates as the compensation signal a phase-shifted oscillation signal component. The control unit is able to calculate the required phase shift depending on the frequency of the oscillation signal component. In many cases, this further implementation requires less computing time than a signal analysis.

According to the invention, the control unit calculates a control signal and a compensation signal. In one embodiment, both signals have a respective pulse width modulation. The pulse width modulation of the control signal has a control signal duty cycle, the pulse width modulation of the compensation signal has a compensation signal duty cycle. A duty cycle is the quotient of the duration of a pulse and the duration of a period. The duty cycle is a value between 0 and 1. The control signal duty cycle depends on a specified value of the setting parameter. The compensation signal duty cycle oscillates, while the control signal duty cycle is preferably constant over time.

Preferably, the control unit controls the or one actuator with a superposition of the control signal and the compensation signal. Preferably, the largest value of the compensation signal duty cycle is smaller than the constant value or the smallest value of the control signal duty cycle. In many cases, this configuration requires relatively little computing time. In many cases, this configuration means that a disturbing oscillation cannot build up (swing up), but is damped and ideally completely prevented.

In one embodiment, the same actuator of the actuator arrangement is controlled by both the control signal and the compensation signal, i.e. generally by a superimposition (overlay) of the control signal on the compensation signal. In this embodiment, the actuator causes both the setting parameter to assume the specified value and the vibration of the valve body to be reduced.

In an alternative embodiment, the actuator arrangement comprises a stronger actuator and a weaker actuator. Both actuators are able to vary the control pressure acting from one side on the valve body. The control unit is able to control the stronger actuator with the control signal and the weaker actuator with the compensation signal. The stronger actuator therefore causes the setting parameter to reach the specified value and the weaker actuator reduces the vibration of the valve body. The variation of the control pressure that the stronger actuator is able to effect is greater than the variation of the control pressure that the weaker actuator is able to effect.

The configuration with the two actuators makes it possible to adapt each actuator to the respective requirements. As a rule, the weaker actuator does not need to change the control pressure over a range as wide as the stronger actuator, but often with a greater frequency and/or more quickly, especially after a vibration is detected.

In a preferred embodiment, the stronger actuator operates pneumatically and the weaker actuator electromagnetically. A preferred implementation of an electromagnetic weaker actuator is described below.

In one implementation, the weaker actuator configured as an electromagnetic actuator comprises a magnetic field generator and an element that can be moved by a magnetic field. Preferably, this movable element, which can be moved by a magnetic field, comprises a magnet, particularly preferably a permanent magnet, and/or an element made of a magnetic material. The magnetic field generator is capable of generating a magnetic field with a strength that varies over space and over time. The movable element is located in this generated magnetic field. The magnetic field causes a force that strives to displace the movable element in the magnetic field and thus relative to the magnetic field generator.

In one embodiment, the movable element is mechanically connected to a component of the valve body, for example to a vibrating diaphragm. Movement of the movable element causes the valve body to move. The magnetic field generator is arranged in such a way that it does not change its position relative to the valve body seat. Preferably, the magnetic field generator is firmly (rigidly) connected to the valve body seat. A movement of the movable element in the magnetic field causes a corresponding movement of the connected component of the valve body. Depending on the magnetic orientation, the magnetic field strives (aims) to move the movable element and thus the valve body away from the valve body seat or towards the valve body or, if the valve body has already reached the valve body seat, to press the valve body against the valve body seat.

Preferably, the movable element is a permanent magnet. When the permanent magnet is connected to the component of the valve body, it is not necessary to electrically contact the permanent magnet. Such electrical contacting would require connecting a movable component with wires or other electrical contacts.

In another embodiment, the magnetic field generator is connected to a component of the valve body and the movable element retains its position relative to the valve body seat.

An embodiment with a stronger actuator and a weaker actuator was described above. Preferably, the stronger actuator is controlled with the control signal, the weaker actuator with the compensation signal. As a rule, the stronger actuator is controlled continuously in order to cause the setting parameter to assume the specified value. The stronger actuator is at least controlled if a change of the setting parameter is required. In one embodiment, the weaker actuator is also permanently active.

In another embodiment, the weaker actuator can be activated and deactivated. The control unit is able to activate the weaker actuator when the vibration of the valve body relative to the valve body seat is greater than a predetermined lower threshold. Preferably, the control unit is able to deactivate the weaker actuator again when the vibration falls below the predetermined lower threshold again. The deactivated weaker actuator is preferably in a resting position (idle position) and is not activated. By activating the weaker actuator with the compensation signal, it is activated and reduces the vibration.

According to this embodiment, the control unit therefore automatically detects the event that the vibration of the valve body is greater than the lower threshold. For this purpose, the ventilation arrangement comprises a vibration sensor, whereby this vibration sensor is configured to measure an indicator of the vibration of the valve body relative to the valve body seat. In one embodiment, the control unit evaluates a signal from this vibration sensor and determines a signal component in this signal that does not result from the control of the stronger actuator with the control signal. The sensor preferably measures the or a setting parameter. A vibration of the valve body leads to an oscillating signal component in a measured indicator for the setting parameter.

According to the invention, the fluid delivery unit of the ventilation arrangement is capable of delivering a gas mixture through the inspiratory fluid guide unit to the patient-side coupling unit. In one embodiment, the fluid delivery unit is part of a ventilator (respirator) for mechanical ventilation of a patient. Preferably, the ventilator performs a sequence of ventilation strokes and ejects in each ventilation stroke a respective quantity of the gas mixture, and the quantity is conveyed to the patient-side coupling unit. It is also possible that another device provides the gas mixture, whereby the other device is connected to at least one supply connection and is located between the or each supply connection and the inspiratory fluid guide unit. The or each supply connection provides at least one component of the gas mixture.

In a preferred embodiment, the control unit is capable of carrying out a closed-loop control. According to this preferred embodiment, the ventilation arrangement comprises at least one setting parameter sensor. The or each setting parameter sensor is capable of measuring an indicator for the or a setting parameter. The control gain (control objective) of the closed-loop control is to ensure that the actual time course of the or one setting parameter follows a predefined required time course. The control deviation should therefore be reduced, ideally to zero. The control of the actuator arrangement with the control signal acts as the control variable (manipulated variable). Vibration of the valve body can act as a disturbance variable (jam signal) and is damped by means of the compensation signal.

According to the invention, the valve arrangement comprises at least one valve. In one embodiment, the valve arrangement comprises at least two valves that are connected in parallel. The two valve body seats of these two valves may have the same diameter or different diameters. In many cases, a relatively low volume flow can be controlled or regulated more easily with two parallel valves than with just one valve. The control unit is able to control each valve with a control signal and a compensation signal, preferably independently of each other. The control unit preferably calculates for each valve a respective control signal and a respective compensation signal.

A second aspect of the invention is described below. The ventilation arrangement according to the second aspect of the invention also comprises a fluid delivery unit, an inspiratory fluid guide unit, a valve arrangement, and an actuator arrangement and preferably a signal processing control unit. The features and advantages of the ventilator arrangement described further above also apply to the ventilator arrangement according to the second aspect. Advantageous embodiments described further above are, where appropriate, also advantageous embodiments of the ventilation arrangement according to the second aspect.

In the following, deviations of the ventilation arrangement according to the second aspect from the ventilation arrangement described above are described.

According to the second aspect of the invention, the actuator arrangement comprises a stronger actuator and a weaker actuator. The valve arrangement comprises a valve, optionally two valves, wherein preferably the two or two valves of the valve arrangement are arranged in parallel to each other. A respective actuator is assigned to the valve or to each of the valves of the valve arrangement. It is possible that both the stronger and the weaker actuator are assigned to the same valve. If the valve arrangement comprises two valves, in one embodiment the stronger actuator is assigned to one valve and the weaker actuator is assigned to the other valve.

Each actuator of the actuator arrangement can contribute to generating the control pressure for the associated valve(s). Optionally, this control pressure is generated exclusively by the assigned actuator or jointly by the at least two assigned actuators. Each actuator is able to vary the control pressure. The stronger actuator is able to vary the control pressure over a larger range (more strongly) than the weaker actuator. This is where the terms “stronger” and “weaker” actuator come from.

In a preferred embodiment, the stronger actuator acts pneumatically, and the weaker actuator acts electromagnetically. In many cases, this configuration allows the stronger pneumatic actuator to vary the control pressure over a wider range, while the weaker electromagnetic actuator is able to adjust the control pressure faster and/or more accurately. This in turn often makes it possible to control an adjustment parameter downstream of the valve assembly both faster and more reliably. The setting parameter is, for example, the volume flow through or the pressure in the second segment.

In one embodiment, the weaker actuator comprises a magnetic field generator and a movable element. The movable element is moved in the magnetic field, whereby this magnetic field is generated by the magnetic field generator. Implementations and advantages of such an actuator have already been described above.

The actuator arrangement with the stronger and the weaker actuator can be used to specifically control a setting parameter of the second segment and, in particular, to perform a closed-loop control for this setting parameter. As described above, the setting parameter describes a pneumatic property of the second segment. The control unit is able to generate a control signal for the stronger actuator and a control signal for the weaker actuator. Both control signals are generated with the control gain that the actual time course of the setting parameter follows a predetermined required time course. The two actuators can be assigned to the same valve, i.e. influence the control pressure of the same valve, or be assigned to two different valves.

In one implementation, the control pressure of one valve is therefore changed with the stronger actuator and the control pressure of the other valve is changed with the weaker actuator. In many cases, this configuration makes it possible to quickly bring the setting parameter close (near) to a desired value, namely with the aid of the stronger actuator, and then to reduce the remaining deviation between the actual and the desired value of the setting parameter, namely with the weaker actuator.

The invention is described below by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of the ventilation arrangement of an embodiment example;

FIG. 2 is a schematic sectional view of a valve in a closed position (left) and in an open position (right) as well as an actuator for the valve body;

FIG. 3 is a schematic view illustrating how the valve body can vibrate relative to the valve body seat;

FIG. 4 is a graph showing a time course of the pressure and a time course of the volume flow with strong oscillations in two different intervals;

FIG. 5 is a graph showing the time course of the volume flow of FIG. 4;

FIG. 6 is a schematic view showing the valve of FIG. 2 as well as an exemplary actuator and associated control of the actuator;

FIG. 7 is a graph showing a duty cycle of a superimposition of a control signal with a compensation signal;

FIG. 8 is a graph showing two amplitude frequency responses of a volume flow resulting from the superimposition of a control signal with a compensation signal;

FIG. 9 is a graph showing an exemplary course of pressure or volume flow in the frequency range;

FIG. 10 is a graph showing the course of FIG. 9 in the time domain;

FIGS. 11a, 11b, 11c and 11d are graphs showing, depending on the amplitude and the phase shift, how well the compensation signal compensates for vibrations of the valve body;

FIG. 12 is a graph showing effects of an incorrectly determined phase shift;

FIG. 13 is a schematic view showing the compensation signal generated by means of feedback of an oscillating signal component of the setting parameter;

FIG. 14 is a schematic sectional view showing an electromagnetic actuator with a coil ring and a ring-shaped permanent magnet;

FIG. 15 is a graph showing a force-displacement diagram for the actuator of FIG. 14;

FIG. 16 is a graph showing the resulting volume flow with pulsed control of the actuator of FIG. 14; and

FIG. 17 is a partially schematic sectional view of a valve arrangement with a pneumatic and an electromagnetic actuator.

### DESCRIPTION OF PREFERRED EMBODIMENTS

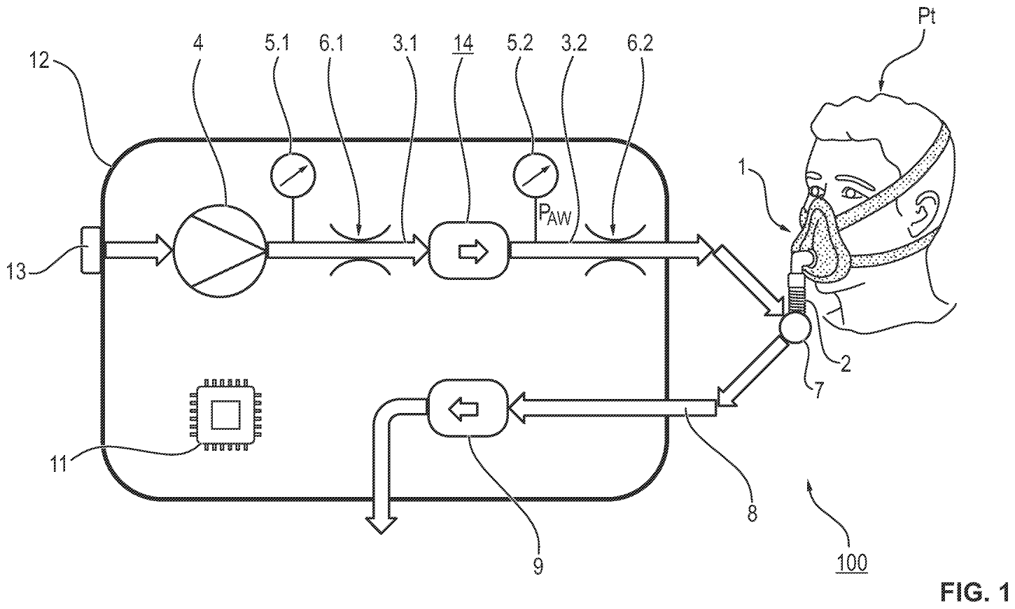

Referring to the drawings, FIG. 1 schematically shows a preferred application of the invention. A patient Pt is artificially ventilated with a ventilation arrangement 100. A patient-side coupling unit is attached to and/or in the body of the patient Pt, in the embodiment example a breathing mask 1 on his/her face. An inspiratory fluid guide unit, for example a tube, comprises two segments 3.1 and 3.2 described below and connects a schematically shown ventilator 12 to a Y-piece 7. The patient-side coupling unit 1 is in fluid connection with the Y-piece 7 via a fluid guide unit 2. An expiratory fluid guide unit 8, for example a further tube, leads from the Y-piece 7 into the environment. An end-expiratory valve 9 is arranged in the expiratory fluid guide unit 8, which valve ensures that a minimum pressure is maintained in the lungs of the patient Pt.

The ventilator 12 comprises a schematically shown supply connection 13 for breathing air and oxygen and optionally for compressed air and/or an anesthetic. The ventilator 12 ejects a gas mixture comprising oxygen. Preferably, the ventilator 12 performs a sequence of ventilation strokes and ejects in each ventilation stroke a respective quantity of the gas mixture. The ejected gas mixture flows through the inspiratory fluid guide unit 3.1, 3.2 to the Y-piece 7 and is inhaled by the patient Pt with the aid of the patient-side coupling unit 1. The air exhaled by the patient Pt flows through the Y-piece 7 and the expiratory fluid guide unit 8 into the environment. It is also possible that a ventilation circuit is implemented, and the air exhaled by the patient Pt flows back to the ventilator 12 through the expiratory fluid guide unit 8.

A fluid delivery unit, for example a blower 4 or a pump or a piston-cylinder unit, generates a volume flow, for example a constant volume flow over time, and a pressure, for example a constant pressure over time. The constant pressure over time is 100 mbar, for example.

It is desirable that the actual time course of a setting parameter follows a predetermined required (target) setting parameter time course. The setting parameter describes a pneumatic property of the second segment 3.2 and thus of the patient-side coupling unit 1. The setting parameter is, in particular, the volume flow through the second segment 3.2 or the pressure in the second segment 3.2. It is possible that two different setting parameters should follow a respective predetermined required time course.

A first pressure sensor 5.1 measures an indicator of the actual pressure P in the first segment 3.1. A second pressure sensor 5.2 measures an indicator of the actual pressure P in the second segment 3.2, preferably the pressure in the patient's airway (pressure in airway, P_{AW}). A first volume flow sensor 6.1 measures an indicator of the actual volume flow through the first segment 3.1. A second volume flow sensor 6.2 measures an indicator of the actual volume flow Vol′ through the second segment 3.2. A signal-processing control unit 11 receives a signal from each of the sensors 5.1, 5.2 and 6.1, 6.2 and controls a valve arrangement 14 described below. The control unit 11 performs closed-loop control with the control objective (control gain) that the actual time course of the volume flow Vol′ through the and/or the pressure P in the second segment 3.2 follows a predetermined required (target) time course.

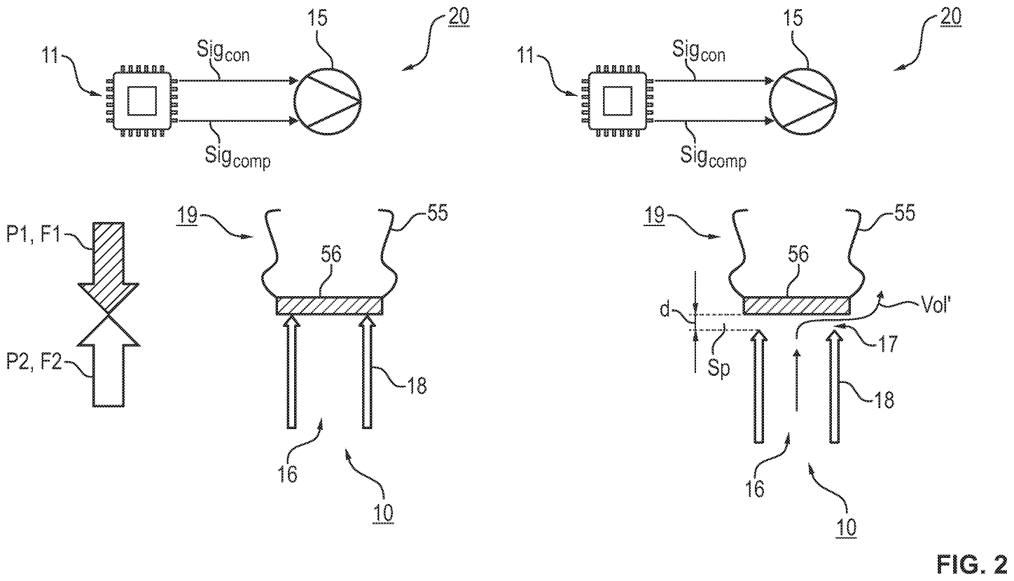

The valve arrangement 14 comprises at least one valve 10, which is schematically shown in FIG. 2. An inlet 16 of the valve 10 is in a fluid connection (fluid communication) with the first segment 3.1 and thus with the fluid delivery unit 4. An outlet 17 of the valve 10 is in a fluid connection with the second segment 3.2 and thus with the patient-side coupling unit 1. Optionally, at least two valves 10 are arranged in parallel.

The valve 10 or each of the valves 10 comprise a respective valve body 19, which in the example shown comprises a resilient diaphragm 56 and a bellows 55, and a valve body seat 18, in the example shown a circular valve body seat (crater) 18. In one embodiment, the diaphragm 56 comprises a rigid plate and a resilient sheathing of this rigid plate. The diaphragm 56 is connected to the elastic bellows 55 in a fluid-tight manner. The valve body 19 can move exclusively linearly relative to the valve body seat 18, up and down in the example shown. In the implementation shown, the diaphragm 56 can move linearly in two opposite directions relative to the valve body seat 18, and the bellows 55 is compressed or relaxes again.

FIG. 2 shows on the left the valve 10 in the closed state, in which the diaphragm 56 is in contact with the valve body seat 18, and on the right the valve 10 in the open state, in which a gap Sp occurs between the valve body seat 18 and the diaphragm 56. A gas flows through the gap Sp. This gap Sp has the thickness (width) d. The arrow Vol′ indicates the volume flow.

A control pressure P1 strives to move the valve body 19 towards the valve body seat 18 and thereby to close the valve 10. An inlet pressure (upstream pressure, pre-pressure) P2 acting in the opposite direction strives to move the valve body 19 away from the valve body seat 18 and thereby to open the valve 10. The inlet pressure P2 is predominantly, usually exclusively, generated by the fluid delivery unit 4. The control pressure P1 is generated by a controllable actuator arrangement 20 with at least one actuator 15 and optionally by a resetting spring force of the elastic bellows 55. The resetting spring force strives to move the bellows 55 into a resting state. The actuator 15 is able, for example, to change the pressure in a control chamber not shown, whereby the pressure in the control chamber acts on the valve body 19 from the side opposite the valve body seat 18 and contributes to the control pressure P1. The control unit 11 is able to actuate the actuator 15 and thereby change the control pressure P1. The inlet pressure P2 can be constant over time and/or depend on a user specification.

Preferably, the control unit 11 applies a given characteristic curve that can be evaluated by a computer, which curve describes the resulting volume flow Vol′ through the outlet 17 or the resulting pressure P at the outlet 17 as a function of the thickness d of the gap Sp between the valve body 19 and the valve body seat 18. The control pressure P1, that can be influenced by the actuator 15, is the manipulated variable of the above-mentioned closed-loop control.

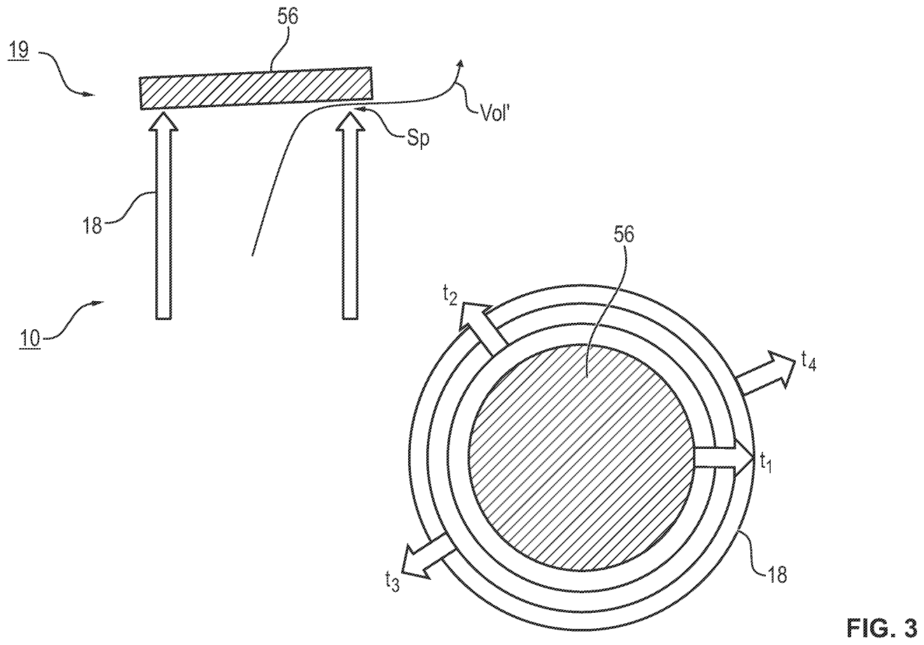

FIG. 2 shows an ideal situation. According to this ideal situation, the gap Sp is either completely closed (left) or has a constant thickness d around its circumference (right). FIG. 3 illustrates a situation that can also occur when using the valve 10. This situation occurs in particular if a relatively small volume flow Vol′ is to be effected through the segment 3.2 and/or if the volume flow Vol′ is to be changed quickly. In addition, the actual geometry of the surface of the valve body 19 and/or the valve body seat 18 often deviates from an ideally flat surface, for example due to unavoidable unevenness.

FIG. 3 shows that the diaphragm 56 does not sit flat (evenly) on the valve body seat 18, but at an angle (slanted). This is shown schematically in FIG. 3 on the left. The circumferential gap Sp therefore has a thickness d that varies around its circumference. The arrow Vol′ indicates the resulting volume flow. FIG. 3 on the right shows a schematic top view on the valve 10. At different successive time points t₁, t₂, t₃, t₄, a respective gap forms between the diaphragm 56 and the valve body seat 18 at four different points.

Therefore, often different flow velocities occur at the same time along the circumference of the gap Sp. As is well known, the so-called Bernoulli effect causes the pressure to drop when the flow velocity of a fluid through a pipe increases. With highly compressible fluids, such as the gas mixture used in artificial ventilation, this Bernoulli effect is relatively strong. In other words, an energy exchange takes place between the pneumatic kinetics (here: volume flow) and the pneumatic potential (here: pressure). The volume flow varies not only over time, but also at a point in time along the circumference of the gap.

In one embodiment, the Bernoulli effect causes a gap to close at one point in time t₁ and a gap to open at another point in time t₂. As a result, the valve body 19 so to speak dances (bounces) on the valve body seat 18.

In general, the energy exchange just described leads to vibrations. These vibrations are often undesirable, particularly for the following reasons:
It is possible that the patient Pt or another human being in the room perceives a noise, whereby the vibrations cause the noise, and the noise is often perceived as unpleasant or even threatening.
The vibrations can cause the volume flow Vol′ and/or the pressure P in the second segment 3.2 to oscillate. Due to these oscillations, in many cases the second volume flow sensor 6.2 is only able to correctly measure the actual volume flow Vol′ and/or the second pressure sensor 5.2 the actual pressure P after a certain settling time (swing-in time). This in turn often means that it takes more time to set a desired volume flow Vol′ and/or a desired pressure P in the second segment 3.2.

As already explained, the situation can arise in which the valve body 19 dances on the valve body seat 18. This undesirable situation can be caused by the pressure oscillating in the segment 3.1 and thus in the inlet 16 of the valve 10. In addition, an oscillating pressure can be reflected between the ventilator 12 and the Y-piece 7.

A boundary condition is that the following procedure is often desired: The valve arrangement 14 with the valve 10 in the inspiratory fluid guide unit 3.1, 3.2 should be controlled and should be controllable with a frequency between 100 Hz and 500 Hz. “Controllability” here means that a control signal has an effect on the control pressure P1. The volume flow Vol′ and/or the pressure P are to be changed with the control signal, and the effect is the actual change in the volume flow Vol′ achieved by the pressure P in the second segment 3.2. In other words: It should be possible to change the volumetric flow Vol′ and/or the pressure P with a step response of less than 0.1 sec.

It would be conceivable to dampen the valve body 19 considerably. In many cases, however, the dampened valve 10 cannot be actuated with the desired high frequency.

The inventors have discovered the following result in internal tests: The vibrations of the valve body 19 relative to the valve body seat 18 are excited by an oscillation of the pressure upstream of the valve arrangement 14, i.e. in segment 3.1. In the unfavorable case, a resonance occurs between the self-generated vibrations in the valve 10 and the oscillations of the pressure in segment 3.1 and/or in segment 3.2. The inventors have found that the most disturbing resonances occur when the pressure in segment 3.1, i.e. upstream of the valve arrangement 14, oscillates at a frequency between 300 Hz and 600 Hz. A lower frequency in segment 3.1 than 300 Hz does not harmonize with the natural frequency (intrinsic frequency) of the system of valve body 19 and valve body seat 18. A higher frequency than 600 Hz leads to many pressure losses and therefore not to a phase-stable superposition.

The inventors have also established the following result in internal tests: When an oscillation system with the valve body 19 and the valve body seat 18 oscillates with resonance, a start-up (ramp-up) behavior of the vibration (oscillation) always occurs. This start-up behavior results from the fact that a vibration is reflected and an increased amplitude results from the reflected vibration. The oscillation system swings up, for which swing-up an amplification is responsible. However, this amplification only occurs to the full extent if the pressure P and volume flow Vol′ remain constant relative to each other. Only in this situation does the next transmission lead to a greater oscillation amplitude.

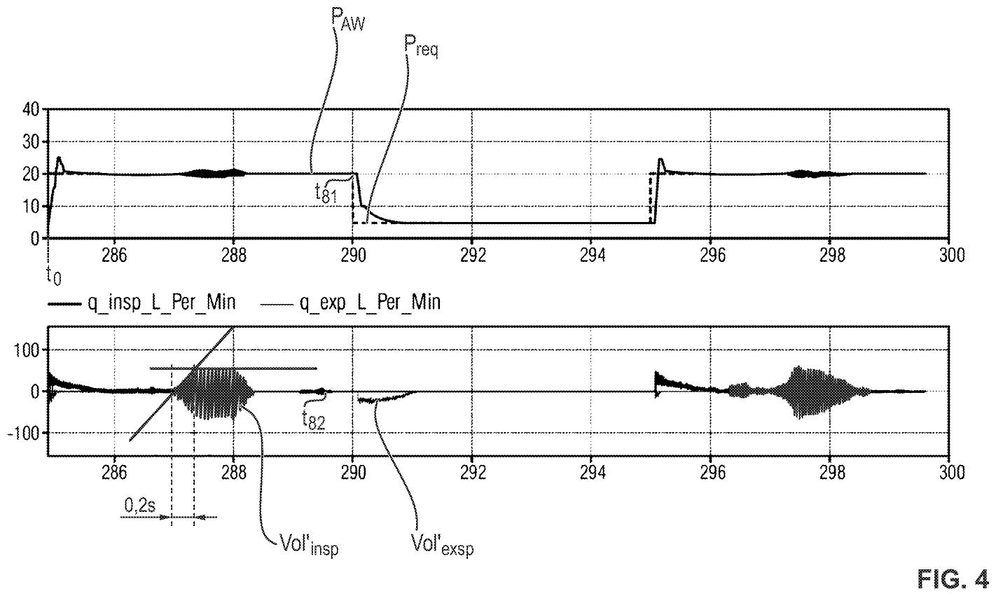

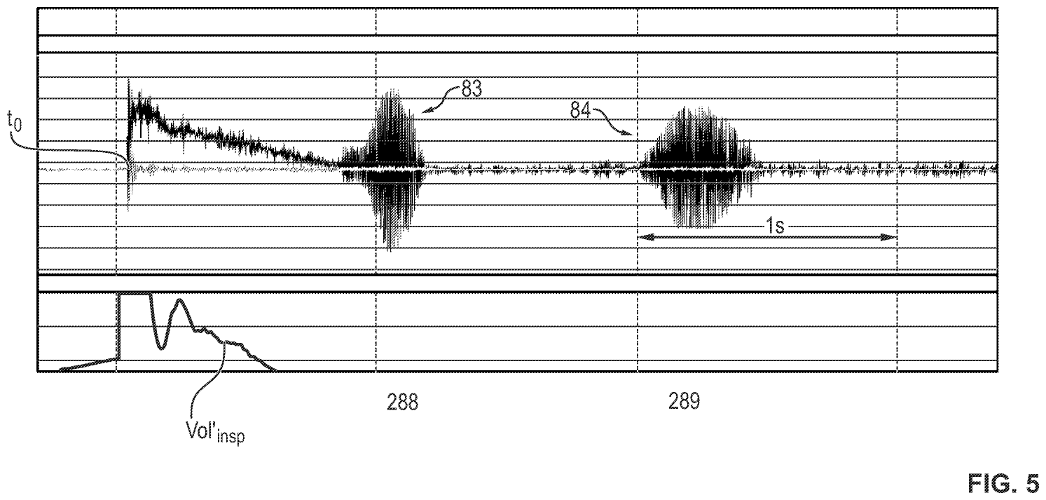

FIG. 4 shows examples of different vibrations. FIG. 5 shows an enlarged section of FIG. 4. The time is plotted on the x-axis, the pressure P in [mbar] in the second segment 3.2 or the volume flow Vol in [l/min] through the segment 3.2 is plotted on the y-axis. The solid line shows the time course of the pressure P_{AW} (pressure in airway), which is the actual pressure in segment 3.2 measured by the second pressure sensor 5.2. The dashed line shows a predetermined target course P_{req} of the pressure P_{AW}. Vol′ᵢₙₛₚ denotes the measured actual volume flow in an inspiratory phase (patient inhales and the gas mixture is directed to the patient-side coupling unit 1), Vol′ₑₓₛₚ denotes the volume flow in an expiratory phase. A positive value indicates a flow of the gas mixture towards the patient-side coupling unit 1, a negative value indicates a flow in the opposite direction. The value shown in FIG. 4 comprises a total of two inspiratory phases and one expiratory phase between the two inspiratory phases. As shown in FIG. 4 above, a pressure is also maintained during an expiratory phase, namely the end-expiratory pressure.

The first inspiration phase begins at time to. This inspiratory phase ends at time t81 and the subsequent expiratory phase begins. A relatively large volume flow Vol′ occurs at the beginning of the first and also the second inspiration phase, which decreases sharply again at around time t=286 and at time t=296. At approximately time t=287 and time t=297, the volume flow begins to oscillate strongly again. The envelope of the amplitude increases over a period of 0.2 seconds. These oscillations approximately in the middle of the first inspiratory phase are associated with small volume flows and are shown in the enlarged representation of FIG. 5 labeled with 83. The pressure P also oscillates during this period. Further oscillations 84 are due to the fact that the airway pressure P_{AW} is readjusted (controlled again) at the end of the first inspiratory phase. Oscillations 84 begin 0.2 sec after the start of oscillations 83. Oscillations 84 take about 60 wave trains and last 0.15 sec. In general, the oscillating volume flow requires at least one wave to oscillate.

As shown in FIG. 2 an actuator 15 influences the control pressure P1, whereby the control pressure P1 acts on the valve body 19 and counteracts the inlet pressure P2. In order to achieve the above-mentioned control objective, the control unit 11 generates a control signal Sig_{con}, which is used to control the actuator 15. One idea of an embodiment of the invention is as follows: The control unit 11 applies a compensation signal Sig_{comp} to the control signal Sig_{con}. The actuator 15 is controlled with a superposition of the control signal Sig_{con} and the compensation signal Sig_{comp}. The compensation signal Sig_{comp} generally causes an additional oscillation of the valve body 19 and thus an additional oscillation of the pressure P in and/or the volume flow Vol′ through the output 17 and the second segment 3.2. The oscillations caused by the compensation signal Sig_{comp} counteract the interfering oscillations, which are shown as examples in FIG. 4 and FIG. 5. Ideally, the application of the compensation signal Sig_{comp} completely prevents the occurrence of interfering oscillations. This ideal case cannot usually be achieved in practice.

One possible implementation is as follows: An unspecific signal with a predefined frequency is applied as the compensation signal Sig_{comp}. This predetermined frequency depends on the maximum frequency with which the system of valve body 19 and valve body seat 18 can still be controlled, i.e. the maximum frequency at which this system still causes a change in the resulting volumetric flow Vol′. Preferably, the specified frequency is at most half as high as the maximum frequency. If this maximum frequency is 300 Hz, for example, a constant signal of 100 Hz or 150 Hz is used as the compensation signal Sig_{comp}.

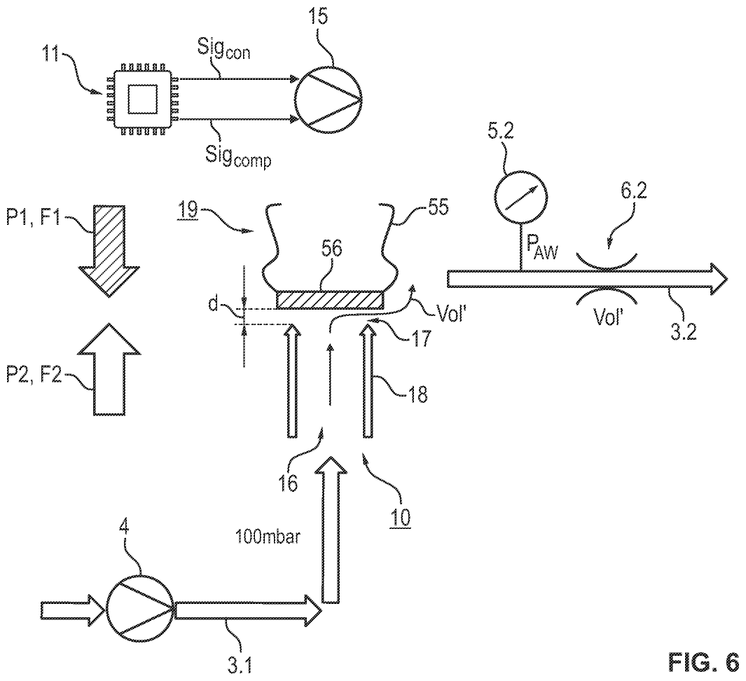

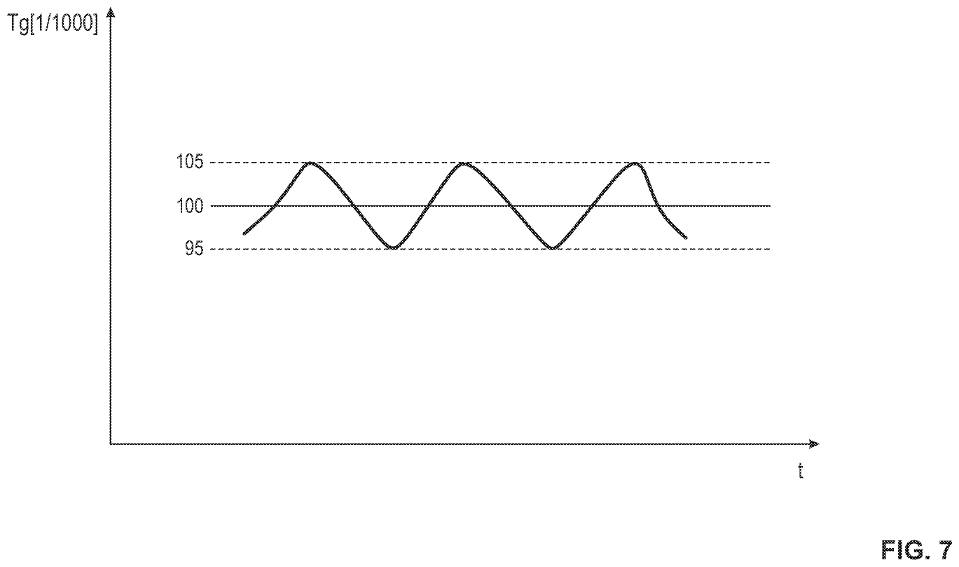

FIG. 6 and FIG. 7 illustrate this configuration using an example. In this example, the actuator 15 is a pump which influences the control pressure P1 acting on the valve body 19. In this example, the pump 15 is able to generate an adjustable volume flow of 0.3 to 0.8 l/min and a pressure of 0.2 to 0.7 mbar. The control signal (DC voltage) Sig_{con} varies between two values, with one value preferably being 0, thus realizing pulse width modulation. The pressure P and/or the volume flow Vol′ generated by the pump 15 depends on the duty cycle (quotient of pulse duration and period duration). For example, the duty cycle of the control signal Sig_{con} is 100/1000, i.e. 10%. The compensation signal Sig_{comp} is also generated by pulse width modulation, whereby the duty cycle varies over time and wherein the average duty cycle is, for example, 10/1000=1%. The compensation signal Sig_{comp} is an alternating voltage with a frequency of 100 Hz and is superimposed on the control signal Sig_{con}. The superimposition provides a pulse width modulation with an oscillating duty cycle.

FIG. 7 shows a schematic diagram, where the time is plotted on the x-axis and the duty cycle of the signal resulting from the superposition of the control signal Sig_{con} with the compensation signal Sig_{comp} is plotted on the y-axis in [1/1000]. As can be seen, the superimposition provides a signal with a duty cycle that varies between 95/1000 and 105/1000 with an effective mean value of 100/1000, whereby the duty cycle of the overall signal resulting from the superimposition oscillates around the effective mean value.

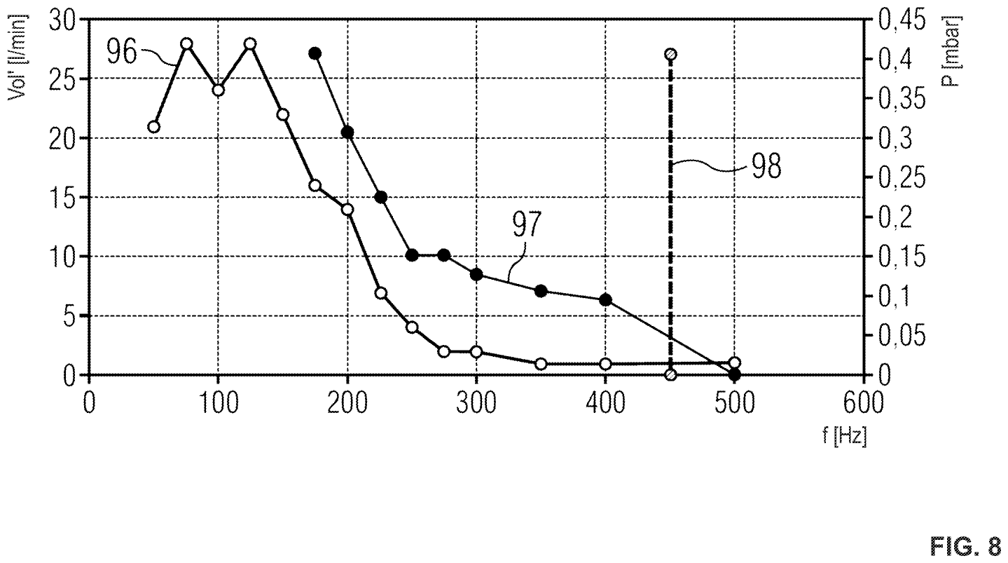

FIG. 8 shows a result when the actuator 15 is controlled with the overall signal just described. The resulting amplitude frequency response 96 of the oscillating volume flow Vol′ through the second segment 3.2 and the resulting amplitude frequency response 97 of the pressure P in the second segment 3.2 are shown. The volume flow Vol′ is measured by the second volume flow sensor 6.2, the pressure P by the second pressure sensor 5.2, cf. FIG. 2 and FIG. 6. The vertical line 98 describes the upper threshold for the frequency of a sound that can still be perceived by the hearing of an adult (hearing threshold).

The embodiment with the non-specific signal is relatively easy to implement, has a broadband effect and is relatively independent of other disturbance variables. However, the sometimes undesirable situation can occur that the frequency of the compensation signal Sig_{comp} is below the hearing threshold of the human ear and therefore a usually low humming noise is audible.

According to a generalization of this embodiment, the frequency of the compensation signal Sig_{comp} is changed during compensation so that a broadband compensation signal Sig_{comp} is applied to the control signal Sig_{con}. This embodiment reduces the risk of static interference forming. One reason for this is that every reflected pressure surge (pressure shock) encounters a different situation. Although the pressure surge is reflected, there is no permanent wave train with which the pressure surge could be superimposed.

In one embodiment, the step of applying the compensation signal Sig_{comp} is triggered when a triggering event has been detected. The triggering event is, for example, the detection of a disturbing oscillation with a relatively large amplitude. Such a disturbing oscillation occurs in the second segment 3.2 and/or in the first segment 3.1. To check whether the triggering event is to occur, at least one of the following pneumatic parameters is measured and evaluated, for example using a Fast Fourier Transformation:
the volume flow Vol′ through the first segment 3.1, which is measured by the first volume flow sensor 6.1,
the pressure P in the first segment 3.1, which is measured by the first pressure sensor 5.1,
the volume flow Vol′ through the second segment 3.2, which is measured by the second volume flow sensor 6.2, or
the pressure P in the second segment 3.2, which is measured by the second pressure sensor 5.2.

In the oscillating signal that describes the pneumatic parameter Vol′, P, a signal component is detected that is not attributable to the control signal Sig_{con} and therefore causes or could cause a disturbing vibration. This is described in more detail below. Preferably, the application of the compensation signal Sig_{comp} is terminated again when the disturbing oscillations have been eliminated.

Preferably, the compensation signal Sig_{comp} is defined in such a way that effectively (statically), i.e. averaged over time, the actual desired valve position is not changed. In other words, the effect of the compensation signal Sig_{comp} on the setting parameter oscillates around the effect of the control signal Sig_{con}. Preferably, the frequency of the compensation signal Sig_{comp} is below the frequency of the actual disturbance (the vibration). This reduces the risk of the disturbance being additionally amplified by a statistically independent asynchronous excitation.

In one embodiment, the amplitude of the broadband compensation signal Sig_{comp} is increased continuously or gradually (iteratively). The process of increasing the amplitude of the compensation signal Sig_{comp} is terminated when the interfering oscillation is sufficiently reduced. Preferably, an upper threshold is set for an amplitude, for example an upper threshold for the amplitude of the compensation signal Sig_{comp} or the amplitude of the resulting pressure P or the resulting volume flow Vol′. This prevents the disturbing oscillation from being reduced, but the compensation signal Sig_{comp} itself leads to audible noise.

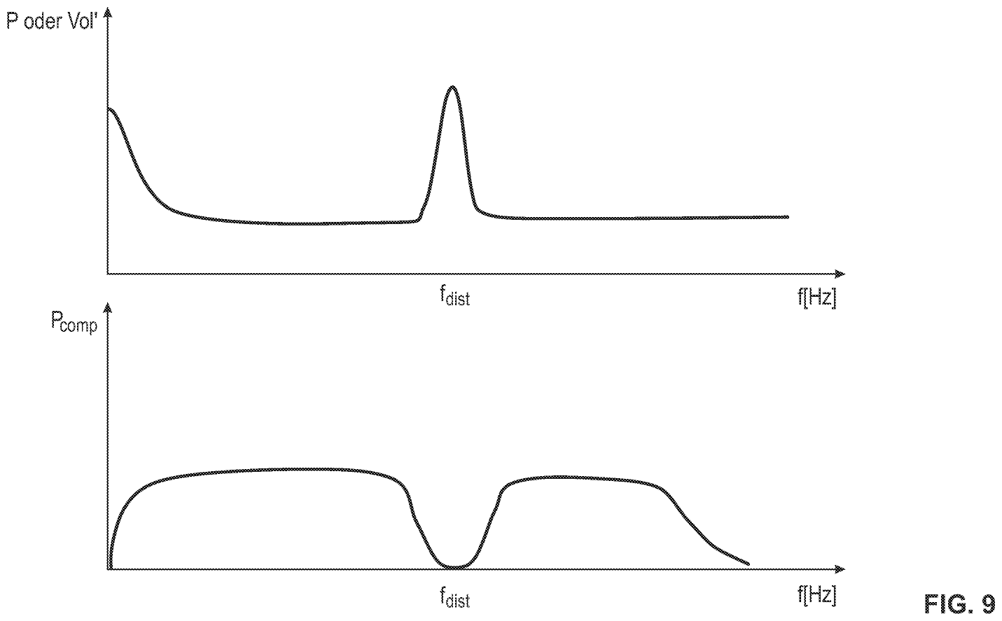

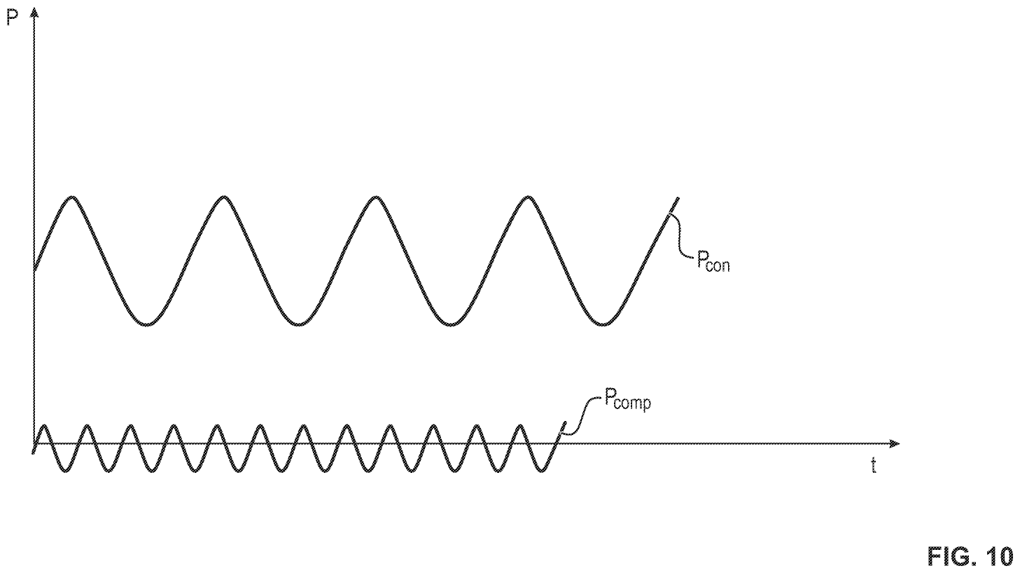

FIG. 9 and FIG. 10 illustrate a different implementation. In FIG. 9 the frequency f is plotted on the x-axis, in FIG. 10 the time t. In the upper diagram of FIG. 9 the pressure P in outlet 17 or the volume flow Vol′ through outlet 17 of valve 10 are plotted on the y-axis. As in FIG. 9 above, an oscillation with a large amplitude occurs around the frequency f_{dist}, which can lead to a disturbing sound. In the lower diagram of FIG. 9 the pressure P_{comp} that the actuator 15 generates due to the control by the compensation signal Sig_{comp} is plotted. Around the frequency f_{dist}, the compensation signal Sig_{comp} causes a lower pressure P_{comp} than at other frequencies. FIG. 10 shows the pressure P on the y-axis. The diagram applies to the frequency f_{dist}. P_{con} is the pressure in the output 17 which results from the control signal Sig_{con} and which causes a disturbing sound. P_{comp} is the pressure resulting from the compensation signal Sig_{comp}. The overall signal causes a pressure course that results from the superimposition of P_{con} with P_{comp}.

A further embodiment is based on determining the frequency of an oscillation that leads or can lead to a resonance. This oscillation occurs in an oscillation system which comprises the valve arrangement 14. The frequency is ideally the resonance frequency, which can be constant over time, but usually changes over time.

In one embodiment, the control unit 11 determines an oscillation of the pressure P and/or the volume flow Vol′ from the time course of the pressure P in and/or the volume flow Vol′ through the second segment 3.2. The control unit 11 automatically determines an oscillation component (signal component) in the oscillations that is not attributable to the fact that the valve arrangement 14 was actuated with the control signal Sig_{con}. This vibration component therefore results from a vibration of the valve body 19 relative to the valve body seat 18. The control unit 11 detects the vibration component resulting from the vibration, for example using a noise filter, a mean value filter or a vibration analysis, for example using Fast Fourier Transformation. The control unit 11 determines the frequency, the amplitude and preferably the phase (phase position, phasing). In practice, the frequency and amplitude change over time.

The control unit 11 preferably sets the amplitude of the compensation signal Sig_{comp} in such a way that a compromise is found between the following two requirements: On the one hand, the compensation signal Sig_{comp} should compensate well for the oscillation component just described. On the other hand, the compensation signal Sig_{comp} should not itself cause excitation of the oscillation system comprising the valve arrangement 20.

In one implementation, the phase position (phase shift) is also determined, i.e. the phase position of the signal component resulting from the vibration relative to the oscillations resulting from the control signal Sig_{con}. In an alternative implementation, several possible phase shifts are specified during operation on a trial base, and the phase shift that leads to the lowest amplitude of the oscillations of the pressure P and/or volume flow Vol′ is selected. This phase shift is preferably stored. The detected phase shift is used, for example, as long as the signal component resulting from the vibrations remains smaller than a predefined threshold.

In many cases, this active negative feedback leads to significantly better suppression of unwanted oscillations. However, greater effort is required for the necessary calculations. To ensure that this configuration avoids unwanted oscillations well, it is often necessary for the actuator 15 to reach the resonant frequencies possible during operation in order to superimpose them. These resonant frequencies can reach up to 600 Hz during artificial ventilation if the control capability is 300 Hz.

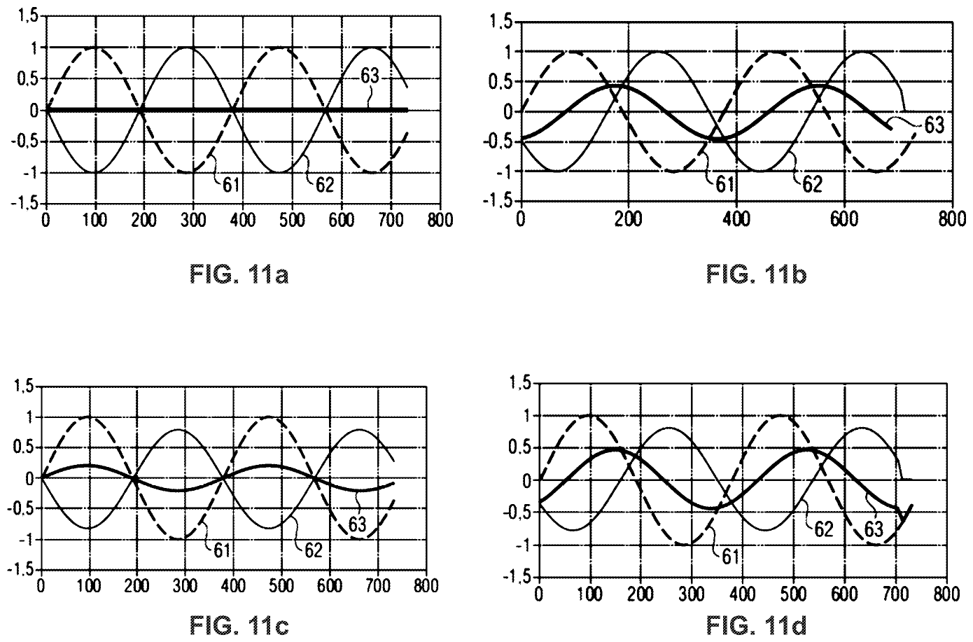

Active negative feedback not only requires the frequency to be determined, but also the amplitude and optionally the phase position. FIGS. 11a-11d present an idealized example to illustrate the importance of correctly detecting the amplitude and phase of an interference signal and generating the compensation signal Sig_{comp} accordingly and then switching it on (applying it). The time is plotted on the x-axis of the four diagrams, the pressure P or the volume flow Vol′ on the y-axis. The frequency was correctly identified in all four examples. The time course of the interference signal is indicated by 61, the time course of the compensation signal by 62 and the time course of a sum signal resulting from the superposition of the interference signal 61 and the compensation signal 62 by 63. In all four examples, the compensation signal 62 is generated in such a way that the compensation signal 62 has a phase shift of 180 degrees relative to the interference signal 61 and the same amplitude, whereby the determined phase and the determined amplitude of the interference signal 61 are used.

In the example of FIG. 11a, the amplitude and the phase position were detected correctly. The compensation signal 62 fully compensates for the interference signal 61. In the example of FIG. 11b, the amplitude was recognized correctly, but not the phase position. In the example in FIG. 11c, the phase position was detected correctly, but the amplitude was too low. In the example in FIG. 11d, an incorrect phase position and too low an amplitude were detected.

It can be seen that even if the amplitude and/or phase position are determined incorrectly, the interference signal can still be sufficiently compensated for in many cases, provided that the error in the determination is not too large.

Preferably, the amplitude and phase position of the compensation signal Sig_{comp} must be constantly checked and adjusted if necessary. This is because an influencing parameter can change rapidly. These influencing parameters can include the temperature, the chemical composition of the gas mixture, the pressure P, and the volume flow Vol′ in segment 6.1. The transfer function of the valve 10 depends on the frequency, but also on the position of the valve body 19 relative to the valve body seat 18 and also on the ambient temperature.

In a variation, the frequency and amplitude are determined, but not the phase position. Instead, the compensation signal Sig_{comp} is varied on a trial basis so that the phase position (phase shift) covers the entire range from 0 degrees to 360 degrees or 0 to 2π. The phase position that leads to a minimum amplitude of the above-mentioned signal component that causes vibrations is then used for the compensation signal Sig_{comp}.

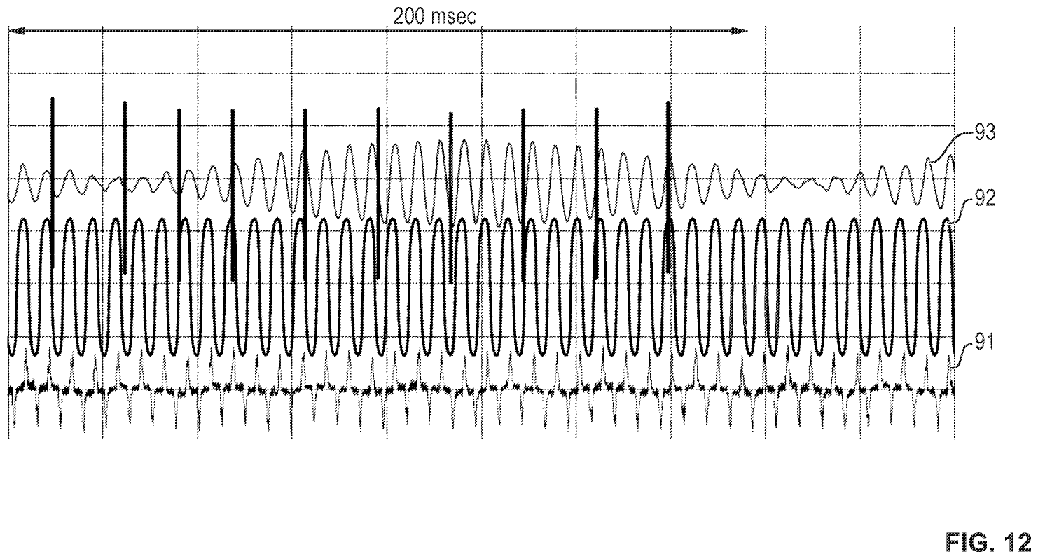

FIG. 12 shows an example in which the frequency of the compensation signal Sig_{comp} does not correspond exactly to the frequency of the vibration component resulting from the vibrations. The course 91 shows the voltage applied to an actuator, whereby this actuator influences the control pressure P1. The actuator can be the pneumatic actuator 15 of FIG. 2 and FIG. 6 or an electromagnetic actuator 58 described below. The time course 92 shows the current through the actuator, which oscillates at a frequency of 205 Hz in the embodiment example shown. The time course 93 shows the oscillating course of the pressure P in the outlet 17 or the volume flow Vol′ through the outlet 17 of the valve 10. The following can be seen: The superposition of the two oscillations caused by the compensation signal Sig_{comp} or by the vibrations results in a beat. The two frequencies of the two oscillations differ only slightly from each other. As can be seen, the beat has an oscillating amplitude. This beat varies between 200 Hz and 202 Hz.

In one embodiment, two different actuators are used, both of which influence the control pressure P2 and thus act on the valve body 19 of the same valve 10. It is also possible that the two actuators act on two different valves, which is described in more detail below. In both embodiments both actuators belong to the actuator arrangement 20 of the embodiment example. One actuator is preferably configured as a pneumatic actuator and particularly preferably comprises a pump or a blower or a piston-cylinder unit. The other actuator acts electromagnetically, wherein preferably a first component of the other actuator is mechanically connected to the valve body 19 and a second component of the other actuator is connected to the valve body seat 18 or at least does not change its position relative to the valve body seat 18. The control unit 11 is able to control the two actuators independently of each other. Preferably, the variation of the control pressure P1 and thus the variation of the pressure P in and/or volume flow Vol′ through the second segment 3.2, which the one and preferably pneumatic actuator 15 can maximally effect, is greater than the variation which the other and preferably electromagnetic actuator 58 can effect.

Preferably, the pneumatic actuator 15 is permanently active and is thus controlled as described above only with the control signal Sig_{con} or with an overall signal, which is a superposition of the control signal Sig_{con} and the compensation signal Sig_{comp}. It is possible that the electromagnetic actuator 58 is also permanently active. It is also possible that the electromagnetic actuator 58 is only activated when a vibration of the valve body 19 relative to the valve body seat 18 is detected, whereby the amplitude of this vibration is greater than a predetermined lower threshold. The control unit 11 detects this vibration, for example, on the basis of a signal from a pressure sensor 5.1, 5.2 and/or a volume flow sensor 6.1, 6.2. In particular, the control unit 11 detects the signal component described above, which the vibrations have caused, and determines the amplitude of this signal component.

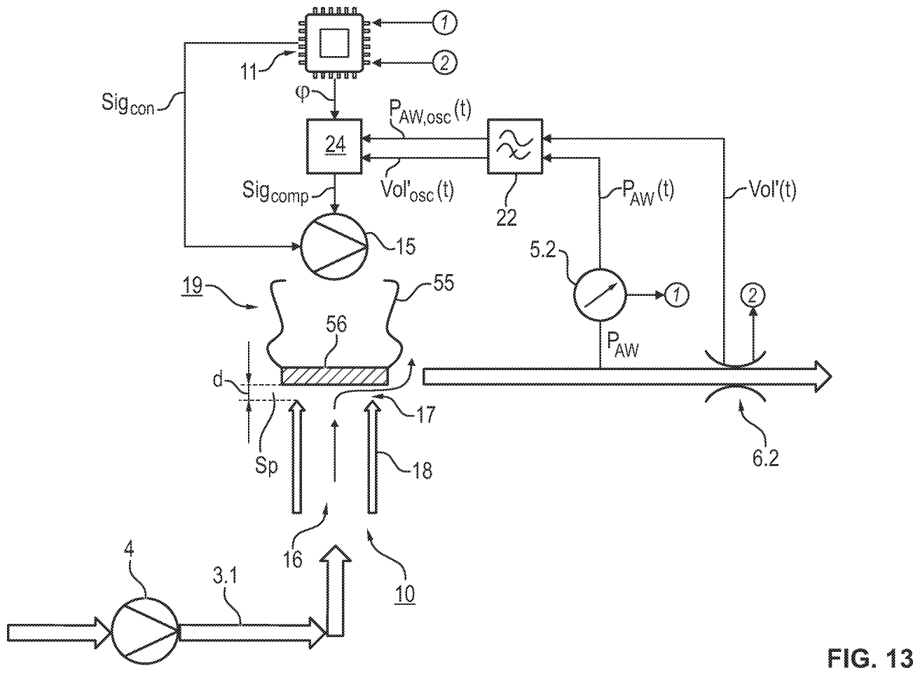

FIG. 13 shows an embodiment for calculating the compensation signal Sig_{comp}. One idea is to feed back the signal of a pressure sensor 5.1, 5.2 and/or a volume flow sensor 6.1, 6.2 and to derive the compensation signal Sig_{comp} from the fed-back signal. In many cases, this embodiment requires less computing time than the embodiments described above, in which the properties of the signal component that leads to the interfering vibrations are analytically determined in a signal from a sensor for a setting parameter.

This idea is realized in the form shown in FIG. 13 as follows: A high-pass filter 22 removes the constant component and the low-frequency component from the signal P_{AW} of the second pressure sensor 5.2, and the remaining oscillating signal component P_{AW,osc} is transmitted to the control unit 11. The control unit 11 calculates the compensation signal Sig_{comp} from the oscillating signal component P_{AW,osc}. The compensation signal Sig_{comp} is generated in such a way that it has the same amplitude as the oscillating signal component P_{AW,osc}(t) and is phase-shifted in relation to the oscillating signal component P_{AW,osc}(t). The required phase shift φ depends on the frequency of the oscillating signal component P_{AW,osc}(t). The control unit 11 determines this frequency and takes into account the flow time required by the gas mixture to flow from the outlet 17 of the valve 10 to the measuring position of the second pressure sensor 5.2. The high-pass filter 22 also removes the constant and low-frequency components from the signal Vol′(t) of the second volume flow sensor 6.2, and the oscillating signal component Vol′_{osc}(t) is transmitted to the control unit 11 and is also used by the control unit 11 to calculate the phase shift φ. The configuration in which a phase shift φ is calculated from the feedback signal Vol′_{osc}(t) and the feedback signal P_{AW,osc}(t) reduces the influence of measurement errors. It is preferable to average the two calculated phase shifts φ.

An electronic component 24 realizes the phase shift φ and supplies the compensation signal Sig_{comp}. Ideally, the vibrations of the valve body 19 are completely eliminated by the feedback.

In lieu of a signal from a sensor 5.2, 6.2, which measures a pneumatic parameter in the second segment 6.2, a signal from a sensor 5.1, 6.1, which measures a pneumatic parameter in the first segment 6.1, can also be fed back. It is also possible to feed back several signals and use a superposition of several fed-back signals as the compensation signal Sig_{comp}.

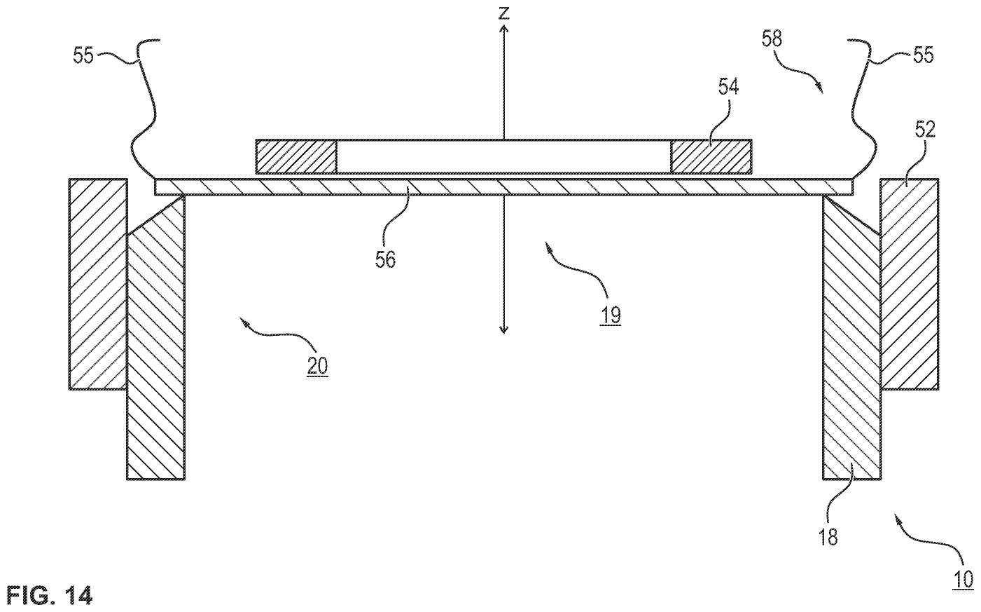

FIG. 14 shows an exemplary embodiment of the above-mentioned electromagnetic actuator 58. The same reference signs have the same meaning as in the previous figures.

The diaphragm 56 of the valve body 19 is mechanically connected to a magnetic ring 54. Preferably, the magnetic ring 54 is configured as a permanent magnet. Preferably, the diaphragm 56 is located between the magnetic ring 54 and the valve body seat 18. Preferably, the magnetic material from which the magnetic ring 54 is made does not comprise iron. This saves mass. The diaphragm 56 is connected to the bellows 55.

A magnetic field generator in the form of a coil ring 52 is guided around the valve body seat 18. The coil ring 52 generates a magnetic field that interacts with the magnetic ring 54. In this implementation, the compensation signal Sig_{comp} is applied to the coil ring 52, and preferentially the strength of the generated magnetic field varies over time and oscillates.

In the positioning and configuration of the electromagnetic actuator 58, the following boundary conditions in particular are preferably taken into account:
The distance between the coil ring 52 and the magnetic ring 54 should be large enough so that no strong force changes occur at the edges. Conversely, however, this distance should not be so large so that the magnetic field generates a sufficiently large effect.
The magnetic field generated should be large enough to move the diaphragm 56 sufficiently far.
On the other hand, a large magnetic field can result in strong gradients, causing the density of the field lines significantly increasing towards the edge. This can have an undesirable consequence: The magnetic ring 54 and thus the diaphragm 56 are moved in a direction at an angle to the Z direction.

For example, the coil ring 52 comprises 280 windings (turns) and has an outer diameter of 23 mm, an inner diameter of 20 mm and a height of 6 mm. Preferably, the number of windings of the coil ring 52 is between 100 and 1000, the outer diameter is between 10 mm and 50 mm, the inner diameter is between 10 mm and 30 mm and the height is between 1 mm and 10 mm. For example, the magnetic ring 54 has an outer diameter of 13 mm, an inner diameter of 9 mm and a height of 1 mm. Preferably, the outer diameter of the magnetic ring 54 is between 5 mm and 30 mm, the inner diameter between 2 mm and 20 mm and the height between 1 mm and 5 mm. Of course, the outer diameter must be larger than the inner diameter.

The diaphragm 56 can move in two opposite directions relative to the valve body seat 18, which is indicated by the arrow labeled Z. In the case of a repelling magnetic force, the magnetic ring 54 and thus the valve body 19 are moved away from the valve body seat 18, i.e. upwards in the illustration. With an attracting magnetic force, the magnetic ring 54 and thus the valve body 19 are moved towards the valve body seat 18. In one embodiment, if neither a repelling nor an attracting force occurs, the valve body 19 is in contact with the valve body seat 18. An attracting force then presses the valve body 19 against the preferably rigid valve body seat 18.

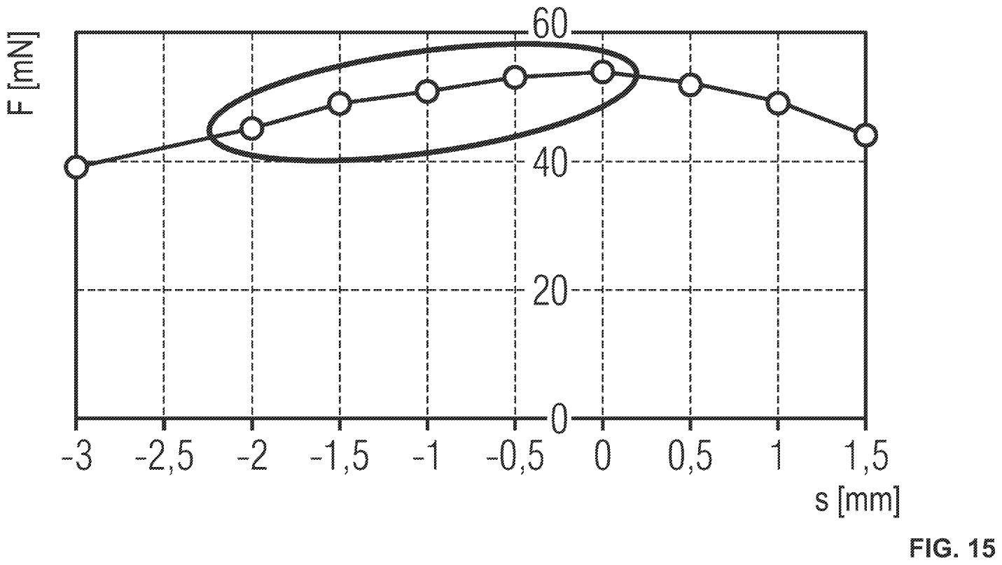

FIG. 15 shows an example of a force-displacement diagram for the arrangement shown in FIG. 14. The distance in the z-direction between the valve body 19 and the valve body seat 18 is shown on the x-axis, and the force exerted by the electromagnetic actuator 58 at this distance is shown on the y-axis. The part of the force-displacement characteristic that is relevant for the movement of the valve body 19 from the zero position (valve body 19 is in contact with the valve body seat 18) is circled. As can be seen, the force that the magnetic field exerts on the permanent magnet 54 is the lower the further away the permanent magnet 54 and thus the diaphragm 56 is from the magnetic field generator 52 and thus from the valve body seat 18. The control pressure P1 counteracts this force. Optionally, when the diaphragm 56 is moved away from the valve body seat 18, the bellows 55 is compressed. The compressed bellows 55 exerts a restoring spring force on the diaphragm 56.

FIG. 14 shows an embodiment in which the permanent magnet 54 is attached to the valve body 19 and the magnetic field generator 52 retains its position relative to the valve body seat 18, i.e. is stationary. Thanks to this configuration, it is not necessary to electrically contact the part of the electromagnetic actuator 58 that is moved together with the valve body 19. The reverse configuration is also conceivable: The magnetic field generator 52 is connected to the valve body 19, and the permanent magnet 54 maintains its position relative to the valve body seat 18.

Instead of a permanent magnet 54, an electrically contacted magnet or an element made of a magnetic material, such as iron or copper, can also be used. In the design with the element made of the magnetic material, the magnetic field is able to attract the element but not repel it.

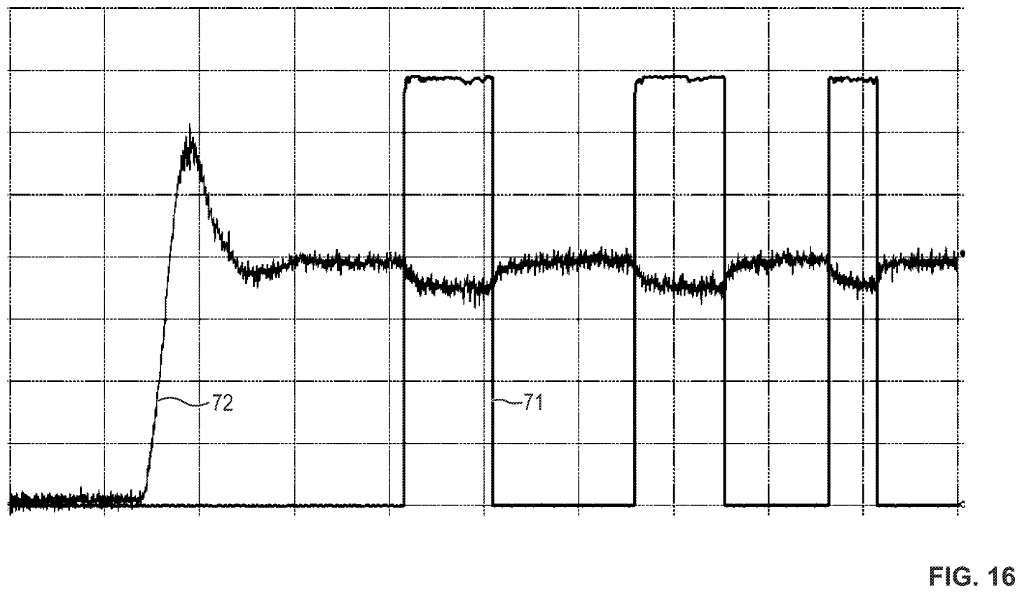

FIG. 16 shows an example of two time courses 71 and 72 which relate to the actuator 52, 54 of FIG. 14 and FIG. 15 and which were obtained in an internal test. The time course 71 describes an indicator of the strength of the current flowing through the coil ring 52. The time course 72 describes the volume flow Vol′ through the outlet 17 of the valve 10. As can be seen, even the maximum possible magnetic field is not sufficient to completely prevent the volume flow Vol′. However, the volume flow Vol′ is clearly reduced. The coil ring 52 is pulsed with 1.5 amperes and around 15 volts and not permanently energized, as permanent energization would place a considerable thermal load on the coil ring 52. A time constant of 20 msec was achieved, which is a similar response time to that response time achieved with the pump 15 of FIG. 2 and FIG. 6.

Preferably, the control unit 11 also continuously determines the frequency and amplitude of the vibrations when the electromagnetic actuator 58 is used.

An embodiment was described above in which the valve arrangement 14 comprises a pneumatically acting actuator 15 and an electromagnetically acting actuator 58. In one embodiment, both actuators 15, 58 contribute to generating the control pressure for the same valve. In another embodiment, the pneumatic actuator 15 is associated with a first valve and the electromagnetic actuator 58 is associated with a second valve. In both embodiments, the pneumatic actuator 15 is preferably able to cause a greater variation in the control pressure than the electromagnetic actuator 58. It is possible that, as described above, the electromagnetic actuator 58 is used to dampen or ideally completely prevent oscillations of the valve body of the associated valve. It is also possible that both the pneumatic actuator 15 and the electromagnetic actuator 58 are used to control or regulate the setting parameter or at least one setting parameter in the second segment 3.2.

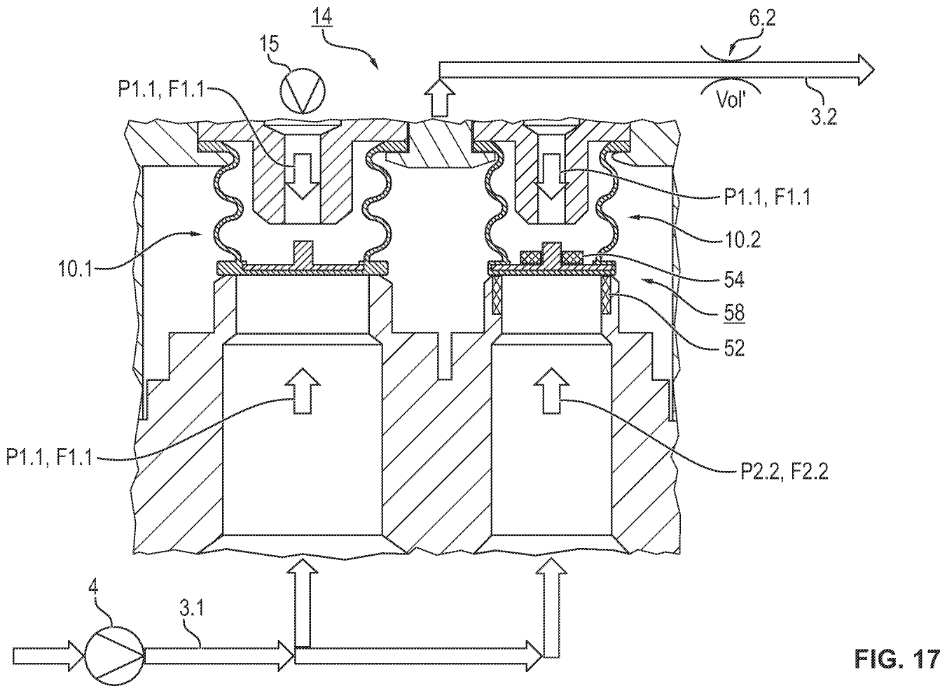

FIG. 17 schematically shows a valve arrangement 14 comprising
two valves 10.1, 10.2 arranged in parallel,
a pneumatic actuator 15 (stronger actuator) and
an electromagnetic actuator 58 (weaker actuator).

In this example, the pneumatic actuator 15 is constructed as described above with reference to FIG. 2 and FIG. 6 and FIG. 13, and the electromagnetic actuator 58 is constructed as described with reference to FIG. 14 and FIG. 15. Each valve 10.1, 10.2 comprises a circular valve body seat and a valve body which is movable relative to the valve body seat. In the implementation shown, the diameter of the circular valve body seat of valve 10.1 is larger than the diameter of the circular valve body seat of valve 10.2. Therefore, the larger valve 10.1 and the smaller valve 10.2 are referred to below. It is also possible that both valve body seats have the same diameter.

The first segment 3.1 generates an inlet pressure (upstream pressure) P2.1 and thus a force F2.1, which acts on the valve body of the larger valve 10.1 from one side. In addition, the first segment 3.1 generates an inlet pressure P2.2 and thus a force F2.2, which acts on the valve body of the smaller valve 10.2 from one side. The pneumatic actuator 15 generates a control pressure P1.1 and thus a force F1.1, which acts on the valve body of the larger valve 10.1 from the other side. The electromagnetic actuator 58 generates a control pressure P1.2 and thus a force F1.2, which acts on the valve body of the smaller valve 10.2 from the other side.

The control unit 11 generates an actuation signal for each of the two valves 10.1, 10.2 and causes both valves 10.1, 10.2 to be actuated with this actuation signal. In the example shown, the two parallel valves 10.1 and 10.2 are both used to control or regulate a setting parameter of the second segment 3.2, for example the volume flow Vol′ through the second segment 3.2 and/or the pressure P_{AW} in the second segment 3.2. In one embodiment, the pneumatic actuator 15 is used to rapidly reduce the deviation between the actual value and a required value of the setting parameter. The electromagnetic actuator 58 is used to further reduce the deviation and ideally bring it to zero. The control unit 11 generates the control signals for the two valves 10.1, 10.2 accordingly.

While specific embodiments of the invention have been shown and described in detail to illustrate the application of the principles of the invention, it will be understood that the invention may be embodied otherwise without departing from such principles.

### LIST OF REFERENCE CHARACTERS

| | |
|---|---|
| 1 | Patient-side coupling unit in the form of a breathing mask, connected to |
| | the fluid guide unit 2 |
| 2 | Fluid guide unit, connects the Y-piece 7 with the patient-side coupling |
| | unit 1 |
| 3.1 | First segment of the inspiratory fluid guide unit, leads from the fluid |
| | delivery unit 4 to the valve arrangement 14 |
| 3.2 | Second segment of the inspiratory fluid guide unit, leads from the valve |
| | arrangement 14 to the Y-piece 7 |
| 4 | Fluid delivery unit in the form of a blower, ejects a gas mixture into the |
| | first segment 3.1, connected to the supply connection 13 |
| 5.1 | First pressure sensor, measures an indicator of the actual pressure in the |
| | first segment 3.1, usually generated by the fluid delivery unit 4 |
| 5.2 | Second pressure sensor, measures an indicator of the actual pressure in |
| | the second segment 3.2, usually the airway pressure P_{AW} |
| 6.1 | First volume flow sensor, measures an indicator of the actual volume |
| | flow through the first segment 3.1 |
| 6.2 | Second volume flow sensor, measures an indicator of the actual volume |
| | flow Vol′ through the second segment 3.2 |
| 7 | Y-piece, connects the fluid guide units 3.2 and 8 on one side with the |
| | fluid guide unit 2 on the other side |
| 8 | Expiratory fluid guide unit, leads from the Y-piece 7 into the |
| | environment |
| 9 | End-expiratory valve in the expiratory fluid guide unit 8 |
| 10 | Valve, comprises the valve body seat 18 and the valve body 19, has the |
| | inlet 16 and the outlet 17, belongs to the valve arrangement 14 |
| 10.1 | Larger valve, has the control pressure P1.1 and the inlet pressure P2.1, is |
| | influenced by pneumatic actuator 15 |
| 10.2 | Smaller valve, with control pressure P1.2 and inlet pressure P2.2, is |
| | affected by electromagnetic element 58 |
| 11 | Signal-processing control unit, receives and processes setting parameter |
| | signals from the sensors 5.1, 5.2 and 6.1, 6.2, controls the actuator |
| | arrangement 20 |
| 12 | Ventilator, comprises the fluid delivery unit 4, the control unit 11, the |
| | valve arrangement 14, the actuator arrangement 20, the sensors 5.1, 5.2 |
| | and 6.1, 6.2, and the supply connection 13 |
| 13 | Supply connection of the ventilator 12, connected to the fluid delivery |
| | unit 4 |
| 14 | Valve arrangement, comprising the valve 10, arranged between the |
| | segments 3.1 and 3.2 |
| 15 | Pneumatic actuator, contributes to the control pressure P1, in one |
| | embodiment has the form of a micropump, acts as the stronger actuator in |
| | one embodiment |
| 16 | Input of valve 10, connected to the first segment 3.1 |
| 17 | Output of valve 10, connected to the second segment 3.2 |
| 18 | Valve body seat in the form of a crater, belongs to the valve 10 |
| 19 | Valve body, comprising the diaphragm 56 and the bellows 55, is part of |
| | the valve 10 |
| 20 | Actuator arrangement, comprising the pneumatic actuator 15 and |
| | optionally the electromagnetic actuator 52, 54 |
| 22 | High-pass filter, filters out the respective constant and low-frequency |
| | components from the oscillating pressure signal P_{AW}(t) and/or the |
| | oscillating volume flow signal Vol′_{osc}(t), provides the oscillating signal |
| | component P_{AW, osc}(t), Vol′_{osc}(t) |
| 24 | Component that generates the compensation signal Sig_{comp} from the |
| | oscillating signal component P_{AW, osc}(t), and/or Vol′_{osc}(t) by means of a |
| | phase shift |
| 52 | Coil ring of an electromagnetic actuator, guided around the valve body |
| | seat 18, generates a magnetic field, belongs to the electromagnetic |
| | actuator 58 |
| 54 | Ring-shaped permanent magnet, mechanically connected to the valve |
| | body 19, is moved in the magnetic field generated by the coil ring 52, |
| | acts as the moving element, belongs to the electromagnetic actuator 58 |
| 55 | Bellows of the valve body 19, carries the diaphragm 56 |
| 56 | The diaphragm of the valve body 19, connected to the bellows 55, is |
| | moved by the actuator arrangement 20 |
| 58 | Electromagnetic actuator, comprising the coil ring 52 and the permanent |
| | magnet 54, acts in one embodiment as the weaker actuator |
| 61 | Interference signal, e.g. vibrations of the valve body 19 |
| 62 | Compensation signal |
| 63 | Sum signal, is a superposition of the interference signal 61 with the |
| | compensation signal 62 |
| 71 | Current through the coil ring 52 |
| 72 | Resulting volume flow through the outlet 17 |
| 83 | Oscillation (vibration) of the valve body 19 in the middle of the first |
| | inspiratory phase |
| 84 | Oscillation (vibration) of the valve body 19 at the end of the first |
| | inspiratory phase |
| 91 | Time course of the voltage with which an actuator 15, 52 is controlled |
| 92 | Time course of the current flowing through an actuator 15, 52 |
| 93 | Time course of the pressure P or the volume flow Vol′ resulting from the |
| | control according to course 91 |
| 96 | Resulting amplitude-frequency response of the volume flow Vol′ through |
| | the segment 3.2 |
| 97 | Resulting amplitude-frequency response of the pressure P in segment 3.2 |
| 98 | Upper threshold for the frequency of a sound that an adult's hearing can |
| | perceive (hearing threshold) |
| 100 | Ventilation arrangement, comprising the ventilator 12, the fluid guide |
| | units 2, 3.1, 3.2, 8, the Y-piece 7, and the patient-side coupling unit 1 |
| d | Thickness of the gap Sp |
| F1 | Force generated by the control pressure P1 on the valve body 19 |
| F1.1 | Force generated by the control pressure P1.1 |
| F2 | Force generated by the inlet pressure P2 on the valve body 19 |
| F2.2 | Force generated by the control pressure P2.2 |
| P_{AW} | Airway pressure (pressure in airway), measured by the second pressure |
| | sensor 5.2 |
| P_{AW}(t) | Pressure signal, describes the time course of the pressure P_{AW} in the |
| | second segment 3.2, generated by the second pressure sensor 5.2, |
| | functions as a setting parameter signal |
| P_{AW, osc}(t) | Oscillating signal component in the signal P_{AW}(t) |
| P_{comp} | Pressure of the output 17 generated by the actuator 15 due to the control |
| | with the compensation signal Sig_{comp} |
| P_{req} | Time-varying required pressure in the second segment 3.2 |
| Pt | Patient, is artificially ventilated, is connected to the patient-side coupling |
| | unit 1 |
| P1 | Control pressure, generated by the actuator 15, 52, 54 and optionally by |
| | an elasticity of the valve body 19 |
| P1.1 | Control pressure at the larger valve 10.1, generated by the pneumatic |
| | actuator 15 |
| P1.2 | Control pressure at the smaller valve 10.1, generated by electromagnetic |
| | actuator 58 |
| P2 | Inlet pressure, generated by the fluid delivery unit 4 and optionally by a |
| | restoring spring force of the bellows 55 |
| P2.1 | Inlet pressure on the larger valve 10.1, generated by fluid delivery unit 4 |
| P2.2 | Inlet pressure on the smaller valve 10.2, generated by fluid delivery unit 4 |
| Sig_{con} | Control signal, influences the control pressure P1, depends on a desired |
| | volume flow Vol′ through the or the pressure P in segment 3.2 |
| Sig_{comp} | Compensation signal, influences the control pressure P1, compensates for |
| | vibrations of the valve body 19 |
| Sp | Gap (slit) between the valve body seat 18 and the valve body 19, has the |
| | thickness d |
| Tg | Duty cycle of a pulse width modulation |
| Vol′ | Volume flow through segment 3.2, measured by the second volume flow |
| | sensor 6.2 |
| Vol′ᵢₙₛₚ | Volume flow in an inspiratory phase |
| Vol′ₑₓₛₚ | Volume flow in an expiratory phase |
| Vol′(t) | Volume flow signal, describes the time course of the volume flow Vol′ |
| | through the second segment 3.2, generated by the second volume flow |
| | sensor 6.2, functions as a setting parameter signal |
| Vol′_{osc}(t) | Oscillating signal component in the signal Vol′(t) |

What is claimed is:

1. A ventilation arrangement for ventilation of a patient via a patient-side coupling unit, the ventilation arrangement comprising:
a fluid delivery unit;
a valve arrangement comprising a valve, wherein the valve comprises a valve body seat and a valve body movable relative to the valve body seat;
an inspiratory fluid guide unit comprising a first segment connecting the fluid delivery unit to the valve arrangement and a second segment connecting the valve arrangement to the patient-side coupling unit;
an actuator arrangement; and
a signal-processing control unit,
wherein a position of the valve body relative to the valve body seat depends on an inlet pressure and on a control pressure and influences a volume flow through the second segment,
wherein the fluid delivery unit is configured to generate a flow of a gas through the first segment and to cause the inlet pressure,
wherein the control unit is configured to generate a control signal and a compensation signal and to control the actuator arrangement with the two generated signals,
wherein the actuator arrangement is configured to contribute to a generation of the control pressure depending on the control signal and on the compensation signal or depending on a superposition of the control signal and the compensation signal,
wherein the control unit is configured to generate the control signal such that a setting parameter describing a pneumatic property of the second segment is brought to or towards a predetermined value based on the control with the control signal, and
wherein the control unit is configured to generate the compensation signal such that vibration of the valve body relative to the valve body seat is completely or at least partially prevented based on actuation with the compensation signal.

2. A ventilation arrangement according to claim 1, wherein the compensation signal oscillates with at least one frequency that lies within a predetermined frequency band.

3. A ventilation arrangement according to claim 1,
wherein an oscillation system, through which gas flows, is comprised by the valve arrangement;
wherein at least one oscillation parameter of the gas flowing through the oscillation system oscillates,
wherein the oscillation of the oscillation parameter comprises an oscillation signal component which results or can result in a vibration of the valve body,
wherein the oscillation of the oscillation signal component has an oscillation signal component frequency and an oscillation signal component amplitude, and
wherein the control unit is configured to generate the compensation signal such that the compensation signal has the oscillation signal component frequency and the oscillation signal component amplitude and is phase-shifted with the oscillation of the oscillation signal component.

4. A ventilation arrangement according to claim 3, further comprising a parameter sensor,
wherein the parameter sensor is configured to measure a pneumatic parameter, the pneumatic parameter being the setting parameter or another pneumatic property of the second segment or a pneumatic property of the first segment,
wherein the parameter sensor is configured to generate a parameter signal which describes a time course of the pneumatic parameter, and
wherein the control unit is configured to generate the compensation signal using the parameter signal such that the compensation signal has the oscillation signal component frequency and the oscillation signal component amplitude and is phase-shifted to the oscillation of the oscillation signal component.

5. A ventilation arrangement according to claim 4, wherein the control unit is configured to determine the signal component oscillation frequency, the signal component oscillation amplitude, and the phase position of the oscillation of the oscillation signal component by a signal analysis of the parameter signal.

6. A ventilation arrangement according to claim 5,
wherein the ventilation arrangement is configured to feedback the parameter signal to the control unit,
wherein the control unit is configured to generate the oscillation signal component by filtering the fed-back parameter signal,
wherein the control unit is configured to use the frequency of the oscillation signal component as the oscillation signal component frequency and to use the amplitude of the oscillation signal component as the oscillation signal component amplitude, and
wherein the control unit is configured to calculate, depending on the oscillation signal component frequency, a phase shift of the compensation signal relative to the feedback oscillation signal component.

7. A ventilation arrangement according to claim 1,
wherein the control unit is configured to generate the control signal as a pulse width modulation control signal with a control signal duty cycle,
wherein the control unit is configured to generate the compensation signal as a pulse width modulation compensation signal with a compensation signal duty cycle, and
wherein the control signal duty cycle depends on a predetermined value of the setting parameter and wherein the compensation signal duty cycle oscillates.

8. A ventilation arrangement according to claim 7, wherein the control unit is configured to control the actuator arrangement with a superposition of the control signal and the compensation signal and wherein a largest value of the compensation signal duty cycle is smaller than a constant or smallest value of the control signal duty cycle.

9. A ventilation arrangement according to claim 1,
wherein the actuator arrangement comprises a stronger actuator and a weaker actuator,
wherein the control unit is configured to control the stronger actuator with the control signal and to control the weaker actuator with the compensation signal,
wherein the stronger actuator is configured to effect a greater variation of the control pressure than the weaker actuator effects.

10. A ventilation arrangement according to claim 9, wherein the stronger actuator is a pneumatically acting actuator, and the weaker actuator is an electromagnetically acting actuator.

11. A ventilation arrangement according to claim 10,
wherein the weaker actuator comprises a magnetic field generator configured to generate a magnetic field and a movable element configured to be movable by the generated magnetic field,
wherein one of the movable element and the magnetic field generator is mechanically connected to a component of the valve body and another one of the movable element and the magnetic field generator is maintained in a position relative to the valve body seat.

12. A ventilation arrangement according to claim 9, further comprising a vibration sensor configured to measure an indicator of a vibration of the valve body relative to the valve body seat,
wherein the weaker actuator is configured to be activated and deactivated, and
wherein the control unit is configured to activate the weaker actuator when the vibration is greater than a predetermined lower threshold, and to deactivate the weaker actuator when the vibration falls below the specified lower threshold.

13. A ventilation arrangement according to claim 1, further comprising:
a setting parameter sensor configured to measure an indicator for the setting parameter and to generate a setting parameter signal which describes the time course of the setting parameter,
wherein the control unit is configured to generate the control signal with the control gain that an actual time course of the setting parameter follows a predetermined required time course, and
wherein the control unit is configured to repeatedly calculate a value for the control signal depending on the setting parameter signal.

14. A ventilation arrangement for ventilation of a patient via a patient-side coupling unit, the ventilation arrangement comprising:
a fluid delivery unit;
a valve arrangement comprising a valve, wherein the valve comprises a valve body seat and a valve body movable relative to the valve body seat;
an inspiratory fluid guide unit comprising a first segment connecting the fluid delivery unit to the valve arrangement and a second segment connecting the valve arrangement to the patient-side coupling unit; and
an actuator arrangement comprising a stronger actuator and a weaker actuator,
wherein a position of the valve body relative to the valve body seat depends on an inlet pressure and on a control pressure and influences a volume flow through the second segment,
wherein the fluid delivery unit is configured to generate a flow of a gas through the first segment and to cause the inlet pressure,
wherein the stronger actuator is assigned to the valve, or the weaker actuator is assigned to the valve, or both the stronger actuator and the weaker actuator are assigned to the valve,
wherein the actuator assigned to the valve, or each actuator assigned to the valve is configured to contribute to a generation of the control pressure for the valve, and
wherein a variation in the control pressure produced by the stronger actuator is greater than a variation in the control pressure produced by the weaker actuator.

15. A ventilation arrangement according to claim 14, wherein the stronger actuator comprises a pneumatic actuator and the weaker actuator comprises an electromagnetically acting actuator.

16. A ventilation arrangement according to claim 15,
wherein the weaker actuator comprises a magnetic field generator and a movable element,
wherein the magnetic field generator is configured to generate a magnetic field and the movable element is movable by the generated magnetic field,
wherein either the movable element is mechanically connected to a component of the valve body and the magnetic field generator is fixed in position relative to the valve body seat or the magnetic field generator is mechanically connected to a component of the valve body and the movable element is fixed in position relative to the valve body seat.

17. A ventilation arrangement according to claim 14, further comprising a signal-processing control unit,
wherein both the stronger actuator and the weaker actuator are assigned to the valve,
wherein the control unit is configured to generate a control signal and a compensation signal and to control the stronger actuator with the control signal and to control the weaker actuator with the compensation signal,
wherein the control unit is configured to generate the control signal such that at least one setting parameter of the second segment is brought to or is brought towards a predetermined value based on the control with the control signal,
wherein the setting parameter describes a pneumatic property of the second segment, and
wherein the control unit is configured to generate the compensation signal such that vibration of the valve body relative to the valve body seat is completely or at least partially prevented due to actuation with the compensation signal.

18. A ventilation arrangement according to claim 14, further comprising:
a signal-processing control unit, and
a setting parameter sensor configured to measure an indicator of a setting parameter and to generate a setting parameter signal describing a time course of the setting parameter,
wherein the control unit is configured to control the time course of the setting parameter such that an actual time course of the setting parameter follows a predetermined target time course, and
to control both the stronger actuator and the weaker actuator depending on the setting parameter signal.

19. A ventilation arrangement according to claim 14,
wherein the valve arrangement comprises a further valve comprising a further valve body seat and a further valve body movable relative to the further valve body seat and
wherein the stronger actuator is assigned to the valve and the weaker actuator is assigned to the further valve.

20. A ventilation process for ventilation of a patient, the process comprising the steps of:
providing a ventilation arrangement, the ventilation arrangement comprising: a fluid delivery unit, an inspiratory fluid guide unit; a valve arrangement comprising a valve; and an actuator arrangement, wherein a first segment of the inspiratory fluid guide unit connects the fluid delivery unit to the valve arrangement and a second segment of the inspiratory fluid guide unit connects the valve arrangement to the patient-side coupling unit, wherein the valve comprises a valve body seat and a valve body movable relative to the valve body seat, wherein a position of the valve body relative to the valve body seat depends on an inlet pressure and a control pressure and wherein the position influences a volume flow through the second segment;
generating a flow of a gas through the first segment that causes the inlet pressure;
generating a control signal;
generating a compensation signal; and
controlling the actuator arrangement with the control signal and the compensation signal, wherein the actuator arrangement contributes to the generation of the control pressure depending on the control signal and on the compensation signal or depending on a superposition of the generated control signal and the generated compensation signal, wherein controlling with the control signal causes to bring a setting parameter describing a pneumatic property of the second segment to or towards a predetermined value and controlling with the compensation signal causes to completely or at least partially prevent vibration of the valve body relative to the valve body seat due to actuation with the compensation signal.
